(19) United States
(12) Patent Application Publication
(10) Pub. No.: US 2003/0109550 A1
(43) Pub. Date: Jun. 12, 2003

(54) Substituted indazole compounds for the treatment of inflammation

(76) Inventors: Michael Clare, Skokie, IL (US); Joyce Z. Crich, Glenview, IL (US); Francis J. Koszyk, Prospect Heights, IL (US); Richard A. Partis, Evanston, IL (US); Michael A. Stealey, Libertyville, IL (US); Richard M. Weier, Lake Bluff, IL (US); Xiangdong Xu, Gurnee, IL (US); Cathleen E. Hanau, Chesterfield, MO (US)

Correspondence Address: PHARMACIA CORPORATION, 800 NORTH LINDBERGH BLVD., MAIL ZONE 04E, ST. LOUIS, MO, 63167, US

(21) Appl. No.: 10/247,388

(22) Filed: Sep. 19, 2002

Related U.S. Application Data
(60) Provisional application No. 60/323,424, filed on Sep. 19, 2001.

(51) Int. Cl. A61K 31/4439; A61K 31/433; A61K 31/4184; A61K 31/4192; A61K 31/416; A61K 31/404

(52) U.S. Cl. 514/338; 514/339; 514/367; 514/375; 514/361; 514/394; 514/406; 514/415; 548/126; 548/152; 548/217; 548/304.7; 548/361.1; 548/465; 514/359

(57) ABSTRACT

The present invention relates to substituted indazole derivatives, compositions comprising such, intermediates, methods of making substituted indazolel derivatives, and methods for treating cancer, inflammation, and inflammation-associated disorders, such as arthritis.

The present application claims priority under Title 35, United States Code, §119 to U.S. Provisional application Serial No. 60/323,424, filed Sep. 19, 2001, which is incorporated by reference in its entirety as if written herein.

### FIELD OF THE INVENTION

The present invention in general is in the field of anti-inflammatory pharmaceutical agents and specifically relates to substituted Indazole derivatives, compositions comprising such, and methods for treating cancer, inflammation, and inflammation-associated disorders, such as arthritis.

### BACKGROUND OF THE INVENTION

The following description of the background of the invention is provided to aid in the understanding the invention, but is not admitted to be or describe prior art to the invention.

NF-κB is a ubiquitous transcription factor that plays a prominent role in the activation of the immune system and in stress responses by regulating the transcription of many early, inducible genes including proinflammatory cytokines, adhesion molecules, growth factors, enzymes, and receptors (Ghosh S., May, M. J., and Kopp. E (1998) Annu. Rev. Immunol. 16, 115-260; Zandi, E., and Karin, M. (1999) Mol. Cell. Biol. 19, 4547-4551; Karin, M. (1999) J. Biol. Chem. 274, 27339-27342). Specificity of gene expression is determined at a cellular level by a diverse array of external stimuli such as bacterial products including LPS, as well as cytokines, most importantly tumor necrosis factor-α (TNFα) and interleukin-β (IL1β). Through the synergistic interaction with other transcription factors, further specificity can be achieved while maintaining enormous potential to coordinately induce a large number of functionally related genes. NF-κB is composed of homo and heterodimers of the Rel protein family and is sequestered in an inactive form in the cytoplasm by members of the IκB family of inhibitory proteins (Ghosh S., May, M. J., and Kopp. E (1998) Annu. Rev. Immunol. 16, 115-260; Zandi, E., and Karin, M. (1999) Mol. Cell. Biol. 19, 4547-4551; Karin, M. (1999) J. Biol. Chem. 274, 27339-27342). IκBs mask the nuclear localization signal on NF-κB, preventing nuclear translocation and hence DNA binding to the promoter regions of responsive genes. Stimulation of cells with an agonist that activates NF-κB leads to a series of biochemical signals, ultimately resulting in the phosphorylation, ubiquitinylation, and degradation of IκBS, thereby releasing NF-κB for nuclear translocation (Ghosh S., May, M. J., and Kopp. E (1998) Annu. Rev. Immunol. 16, 115-260; Zandi, E., and Karin, M. (1999) Mol. Cell. Biol. 19, 4547-4551; Karin, M. (1999) J. Biol. Chem. 274, 27339-27342). Recently, two IκB kinases (IKK1 or IKKα and IKK2 or IKKβ), which phosphorylate IκBs and thereby initiate their degradation, have been cloned and characterized by a number of laboratories (Ghosh S., May, M. J., and Kopp. E (1998) Annu. Rev. Immunol. 16, 115-260; Zandi, E., and Karin, M. (1999) Mol. Cell. Biol. 19, 4547-4551; Karin, M. (1999) J. Biol. Chem. 274, 27339-27342). The catalytic subunits, IKK1 and IKK2, are similar structurally as well as enzymatically and exist as a heterodimer in a large protein complex referred to as the IKK signalsome (Regnier, C., Song, H., Gao, X., Goeddel, D., Cao, Z. and Rothe, M. (1997) Cell 90, 373-383; DiDonato, J. A., Hayakawa, M., Rothwarf, D. M., Zandi, E. and Karin, M. (1997) Nature 388, 548-554; Mercurio, F., Zhu, H., Murray, B. W., Shevchenko, A., Bennett, B. L., Li, J. W., Young, D. B., Barbosa, M., Mann, M., Manning, A. and Roa, A. (1997) Science 278, 860-866; Zandi, E. Rothwarf, D. M., Delhase, M., Hayadawa, M and Karin, M. (1997) Cell 91, 243-252; Woronicz, J. D., Gao, X., Cao, Z., Rothe, M. And Goeddel, D. V. (1997) Science 278, 866-869). A third protein, NEMO (IKKγ, IKKAP1), is a regulatory adapter protein necessary for IKK activation and kinase activity (Yamaoka, S., Courtois, G., Bessia, C., Whiteside, S. T., Weil, R., Agou, F., Kirk, H. E., Kay, R. J., and Ireal, A. (1998) Cell 93, 1231-1240; Rothwarf, D. M., Zandi, E., Natoli, G., Karin, M. (1998) Nature 395, 297; Mercurio, F., Murray, B. W., Shevchenko, A., Bennet, B. L., Young, D. B., Li, J. W., Pascual, G., Motiwala, A., Zhu, H., Mann, M and Manning, A. M. (1999) Mol. Cell. Biol 2, 1526-1538). IKK1 and IKK2 are co-expressed in most human adult tissues as well as in different developmental stages of mouse embryos (Regnier, C., Song, H., Gao, X., Goeddel, D., Cao, Z. and Rothe, M. (1997) Cell 90, 373-383; DiDonato, J. A., Hayakawa, M., Rothwarf, D. M., Zandi, E. and Karin, M. (1997) Nature 388, 548-554; Mercurio, F., Zhu, H., Murray, B. W., Shevchenko, A., Bennett, B. L., Li, J. W., Young, D. B., Barbosa, M., Mann, M., Manning, A. and Roa, A. (1997) Science 278, 860-866; Zandi, E. Rothwarf, D. M., Delhase, M., Hayadawa, M and Karin, M. (1997) Cell 91, 243-252; Woronicz, J. D., Gao, X., Cao, Z., Rothe, M. and Goeddel, D. V. (1997) Science 278, 866-869; Hu, M. C. T., and Wang, Y. (1998) Gene 222, 31-40). This kinase complex appears to represent a critical, common denominator in the activation of NF-κB in a number of signal transduction pathways stimulated by a variety of agonists including cytokines, such as TNFα and IL1β, microbial products such as LPS and viral proteins such as TAX, as well as phorbol esters, oxidizing agents and serine/tyrosine phosphatases (Ghosh S., May, M. J., and Kopp. E (1998) Annu. Rev. Immunol. 16, 115-260; Zandi, E., and Karin, M. (1999) Mol. Cell. Biol. 19, 4547-4551; Karin, M. (1999) J. Biol. Chem. 274, 27339-27342).

IKK1 (also termed IKKα, Regnier, C., Song, H., Gao, X., Goeddel, D., Cao, Z. and Rothe, M. (1997) Cell 90, 373-383; DiDonato, J. A., Hayakawa, M., Rothwarf, D. M., Zandi, E. and Karin, M. (1997) Nature 388, 548-554; Mercurio, F., Zhu, H., Murray, B. W., Shevchenko, A., Bennett, B. L., Li, J. W., Young, D. B., Barbosa, M., Mann, M., Manning, A. And Roa, A. (1997) Science 278, 860-866) was cloned simultaneously by standard biochemical purification of the IκB kinase activity from TNFα stimulated HeLa S3 cells and by its interaction with the MAP3K, NF-κB inducing kinase (NIK), in a yeast two-hybrid screen. IKK1 was identified as the previously cloned serine-threonine kinase, CHUK (Connelly, M. and Marcu, K. (1995) Cell. Mol. Biol. Res. 41, 537-549). IKK1 (also termed IKKα) is an 85 kDa, 745 amino acid protein that contains an N-terminal serine/threonine kinase catalytic domain, a leucine zipper-like amphipathic helix, and a C-terminal helix-loop-helix domain. IKK2 (also termed IKKβ) was also cloned by standard biochemical purification, copurifying with IKK1 from TNFα stimulated HeLa S3 cells as well as by being identified in the public database from an EST clone with sequence homology to IKK1 (Mercurio, F., Zhu, H., Murray, B. W., Shevchenko, A., Bennett, B. L., Li, J. W., Young, D. B., Barbosa, M., Mann, M., Manning, A. and Roa, A. (1997) Science 278, 860-866; Zandi, E. Rothwarf, D. M., Delhase, M., Hayadawa, M and Karin, M. (1997) Cell 91, 243-252; Woronicz, J. D., Gao, X., Cao, Z., Rothe, M. And Goeddel, D. V. (1997) Science 278, 866-869). IKK2 is an 87 kDa, 756 amino acid protein with the same over all topology as IKK1 except for the addition of an 11 amino acid extension at the C-terminus. IKK1 and IKK2 are 52% identical overall with 65% identity in the kinase domain and 44% identity in the protein interaction domains in the C-terminus. Data obtained using transient mammalian expression analysis, by in vitro translation experiments and by coexpression in a baculoviral system reveals that IKK1 and IKK2 associate preferentially as a heterodimer through their leucine zipper motifs. Although homodimers have also been described in these systems, the heterodimer is thought to be the physiologic form of the kinase in mammalian cells (Zandi, E. Rothwarf, D. M., Delhase, M., Hayadawa, M and Karin, M. (1997) Cell 91, 243-252; Li, J., Peet, G. W., Pullen, S. S., Schembri-King, J., Warren, T. C., Marcu, K. B., Kehry, M. R., Barton, R. and Jakes, S. (1998) J. Biol. Chem. 273, 30736-30741). Finally, NEMO (also termed IKKγ) contains three α-helical regions including a leucine zipper, interacts preferentially with IKK2 and is required for activation of the heterodimeric kinase complex perhaps by bringing other proteins into the signalsome complex (Yamaoka, S., Courtois, G., Bessia, C., Whiteside, S. T., Weil, R., Agou, F., Kirk, H. E., Kay, R. J., and Ireal, A. (1998) Cell 93, 1231-1240; Rothwarf, D. M., Zandi, E., Natoli, G., Karin, M. (1998) Nature 395, 297; Mercurio, F., Murray, B. W., Shevchenko, A., Bennet, B. L., Young, D. B., Li, J. W., Pascual, G., Motiwala, A., Zhu, H., Mann, M and Manning, A. M. (1999) Mol. Cell. Biol. 2, 1526-1538).

The kinase activities of IKK1 and IKK2 are regulated by phosphorylation and require an intact leucine zipper (LZ) for dimerization as well as an intact helix-loop-helix (HLH) domain, which can exert a positive regulatory effect on kinase activity even when it is expressed in trans with the remainder of the IKK protein (Regnier, C., Song, H., Gao, X., Goeddel, D., Cao, Z. and Rothe, M. (1997) Cell 90, 373-383; DiDonato, J. A., Hayakawa, M., Rothwarf, D. M., Zandi, E. and Karin, M. (1997) Nature 388, 548-554; Mercurio, F., Zhu, H., Murray, B. W., Shevchenko, A., Bennett, B. L., Li, J. W., Young, D. B., Barbosa, M., Mann, M., Manning, A. and Roa, A. (1997) Science 278, 860-866; Zandi, E. Rothwarf, D. M., Delhase, M., Hayadawa, M and Karin, M. (1997) Cell 91, 243-252; Woronicz, J. D., Gao, X., Cao, Z., Rothe, M. and Goeddel, D. V. (1997) Science 278, 866-869; Dehase, M., Hayakawa, M., Chen, Y., and Karin, M. (1999) Science 284, 309-313). Both IKK subunits contain a canonical MAPKK activation loop motif near the N-terminus which is the target for phosphorylation and activation of kinase activity by MAP3Ks such as NIK and MEKK1, although the physiologic regulation by these two upstream kinases awaits further characterization (Zandi, E., and Karin, M. (1999) Mol. Cell. Biol. 19, 4547-4551; Karin, M. (1999) J. Biol. Chem. 274, 27339-27342; Karin, M., and Delhase, M. (1998) Proc. Natl. Acad. Sci. USA 95, 9067-9069). Finally, phosphorylation of serines in the C-terminus of IKK2 results in a decrease in IKK activity and it is postulated to be responsible for the transient kinase activity seen after stimulation of cells with an agonist (Dehase, M., Hayakawa, M., Chen, Y., and Karin, M. (1999) Science 284, 309-313).

IKK2 demonstrates a more potent kinase activity compared to IKK1 using IκBα or IκBβ as a substrate (Mercurio, F., Zhu, H., Murray, B. W., Shevchenko, A., Bennett, B. L., Li, J. W., Young, D. B., Barbosa, M., Mann, M., Manning, A. and Roa, A. (1997) Science 278, 860-866; Zandi, E. Rothwarf, D. M., Delhase, M., Hayadawa, M and Karin, M. (1997) Cell 91, 243-252; Woronicz, J. D., Gao, X., Cao, Z., Rothe, M. and Goeddel, D. V. (1997) Science 278, 866-869; Dehase, M., Hayakawa, M., Chen, Y., and Karin, M. (1999) Science 284, 309-313). Mutations of the phospho-acceptor serine residues within the MAPKK activation loop alters IKK2 kinase activity; the serine to alanine substitutions result in decreased kinase activity whereas the serine to glutamic acid substitutions result in a constitutively active kinase. Similar alanine mutations in IKK1 do not result in a decreased stimulation of total IKK activity in response to TNFα or IL1β (Dehase, M., Hayakawa, M., Chen, Y., and Karin, M. (1999) Science 284, 309-313). IKK2 being the dominant kinase activity within the IKK complex is further supported by the analysis of fibroblasts from mice deficient in IKK1 or IKK2. Fibroblasts lacking IKK1 retain full IKK activity in response to cytokines and could activate NF-κB. In contrast, fibroblasts lacking IKK2 do not exhibit IKK activity when stimulated with cytokines nor do they activate NF-κB. Furthermore, the phenotypes of each IKK knock out is unique with IKK1 deficiency resulting in skin and skeletal defects and IKK2 knock out being embryonic lethal due to hepatocyte apoptosis (Li, Q., Antwerp, D. V., Mercurio, F., Lee, K., and Verma, I. M. (1999) Science 284, 321-325; Takeda, K., Tekeuchi, O., Tsujimura, T., Itami, S., Adachi, O., Kawai, T., Sanjo, H., Yoshikawa, K., Terada, N, and Akira, S. (1999) Science 284, 313-316; Hu, Y., Baud, V., Delhase, M., Zhang, P., Deerinck, T., Ellisman, M., Johnson, R., and Karin, M. (1999) Science 284, 315-320; Li, Q., Lu, Q., Hwang, J. Y., Buscher, D., Lee, K., Izpisua-Belmonte, J. C., and Verma, I. M. (1999) Gene and Development 13, 1322-1328; Tanaka, M., Fuentes, M. E., Yamaguchi, K., Durnin, M. H., Dalrymple, S. A., Hardy, K. L., and Goeddel, D. V. (1999) Immunity 10, 421-429).

It is well-known that NF-KB plays a key role in the regulated expression of a large number of pro-inflammatory mediators including cytokines such as IL-6 and IL-8, cell adhesion molecules, such as ICAM and VCAM, and inducible nitric oxide synthase (iNOS). Such mediators are known to play a role in the recruitment of leukocytes at sites of inflammation and in the case of iNOS, may lead to organ destruction in some inflammatory and autoimmune diseases. The importance of NF-κB in inflammatory disorders is further strengthened by studies of airway inflammation including asthma in which NF-κB has been shown to be activated. This activation may underlie the increased cytokine production and leukocyte infiltration characteristic of these disorders. In addition, inhaled steroids are known to reduce airway hyper responsiveness and suppress the inflammatory response in asthmatic airways. In light of the recent findings with regard to glucocorticoid inhibition of NF-κB, one may speculate that these effects are mediated through an inhibition of NF-κB. Further evidence for a role of NF-κB in inflammatory disorders comes from studies of rheumatoid synovium. Although NF-κB is normally present as an inactive cytoplasmic complex, recent immunohistochemical studies have indicated that NF-κB is present in the nuclei, and hence active, in the cells comprising rheumatoid synovium. Furthermore, NF-κB has been shown to be activated in human synovial cells in response to stimulation with TNF-α. Such a distribution may be the underlying mechanism for the increased cytokine and eicosanoid production characteristic of this tissue. See Roshak, A. K., et al., J. Biol. Chem., 271, 31496-31501 (1996).

The NF-κB/Rel and IκB proteins are also likely to play a key role in neoplastic transformation. Family members are associated with cell transformation in vitro and in vivo because of overexpression, gene amplification, gene rearrangements, or translocations (Gilmore T D, Trends Genet 7:318-322, 1991; Gillmore T D, Oncogene 18:6925-6937, 1999; Rayet B. et al., Oncogene 18: 6938-6947, 1991). In addition, rearrangement and/or amplification of the genes encoding these proteins are seen in 20-25% of certain human lymphoid tumors. In addition, a role for NF-κB in the regulation of apoptosis, cell cycle progression, invasion, and metastasis has been reported (Bours V. et al., Biochemical Pharmacology 60:1085-1090, 2000) strengthening the role of this transcription factor in the control of cell proliferation. The inhibition of NF-κB has been shown to potentiate TNF- and cancer therapy through increased apoptosis (Wang C-Y et al., Science 274:784-787, 1996; Wang C-Y et al., Nat Med 5:412-417, 1999). It has also been shown that human T-cell leukemia virus type 1 (HTLV1) infected cells (the etiological agent of an aggressive malignancy of activated CD4⁺ T lymphocytes), IKKα and IKKβ are expressed constitutively, which normally function in a transient manner (Chu Z-L et al., J of Biological Chemistry 273:15891-15894, 1998). The HTLV1 transforming and transactivating protein (Tax) has been shown to bind MEKK1 and increases the activity of IKKβ to enhance phosphorylation of serine residues in IκBα that lead to its degradation.

Pyrazoles have been described for use in the treatment of inflammation. U.S. Pat. No. 5,134,142 to Matsuo et al describes 1,5-diaryl pyrazoles, and specifically, 1-(4-fluorophenyl)-5-[4-(methylsulfonyl)phenyl]-3-trifluoromethyl pyrazole, as having anti-inflammatory activity.

U.S. Pat. No. 3,940,418 to R. Hamilton describes tricyclic 4,5-dihydrobenz[g]indazoles as anti-inflammatory agents. In addition, R. Hamilton [J. Heterocyclic Chem., 13, 545 (1976)] describes tricyclic 4,5-dihydrobenz[g]indazoles as anti-inflammatory agents. U.S. Pat. No. 5,134,155 describes fused tricyclic pyrazoles having a saturated ring bridging the pyrazole and a phenyl radical as HMG-CoA reductase inhibitors. European publication EP 477,049, published Mar. 25, 1992, describes [4,5-dihydro-1-phenyl-1H-benz[g]indazol-3-yl]amides as having antipsychotic activity. European publication EP 347,773, published Dec. 27, 1989, describes [4,5-dihydro-1-phenyl-1H-benz[g]indazol-3-yl]propanamides as immunostimulants. M. Hashem et al [J. Med. Chem., 19, 229 (1976)] describes fused tricyclic pyrazoles, having a saturated ring bridging the pyrazole and a phenyl radical, as antibiotics.

Certain substituted pyrazolyl-benzenesulfonamides have been described in the literature as synthetic intermediates. Specifically, 4-[5-(4-chlorophenyl)-3-phenyl-1H-pyrazol-1-yl]benzenesulfonamide has been prepared from a pyrazoline compound as an intermediate for compounds having hypoglycemic activity [R. Soliman et al, J. Pharm. Sci., 76, 626 (1987)]. 4-[5-[2-(4-Bromophenyl)-2H-1,2,3-triazol-4-yl]-3-methyl-1H-pyrazol-1-yl]benzenesulfonamide has been prepared from a pyrazoline compound and described as potentially having hypoglycemic activity [H. Mokhtar, Pak. J. Sci. Ind. Res., 31, 762 (1988)]. Similarly, 4-[4-bromo-5-[2-(4-chlorophenyl)-2H-1,2,3-triazol-4-yl]-3-methyl-1H-pyrazol-1-yl]benzenesulfonamide has been prepared [H. Mokhtar et al, Pak. J. Sci. Ind. Res., 34, 9 (1991)].

The phytotoxicity of pyrazole derivatives is described [M. Cocco et al, Il. Farmaco-Ed. Sci., 40, 272 (1985)], specifically for 1-[4-(aminosulfonyl)phenyl]-5-phenyl-1H-pyrazole-3,4-dicarboxylic acid.

The use of styryl pyrazole esters for antidiabetes drugs is described [H. Mokhtar et al, Pharmazie, 33, 649-651 (1978)]. The use of styryl pyrazole carboxylic acids for antidiabetes drugs is described [R. Soliman et al, Pharmazie, 33, 184-5 (1978)]. The use of 4-[3,4,5-trisubstituted-pyrazol-1-yl]benzenesulfonamides as intermediates for sulfonylurea anti-diabetes agents is described, and specifically, 1-[4-(aminosulfonyl)phenyl]-3-methyl-5-phenyl-1H-pyrazole-4-carboxylic acid [R. Soliman et al, J. Pharm. Sci., 72, 1004 (1983)]. A series of 4-[3-substituted methyl-5-phenyl-1H-pyrazol-1-yl]benzenesulfonamides has been prepared as intermediates for anti-diabetes agents, and more specifically, 4-[3-methyl-5-phenyl-1H-pyrazol-1-yl]benzenesulfonamide [H. Feid-Allah, Pharmazie, 36, 754 (1981)]. In addition, 1-(4-[aminosulfonyl]phenyl)-5-phenylpyrazole-3-carboxylic acid has been prepared from the above described 4-[3-methyl-5-phenyl-1H-pyrazol-1-yl]benzenesulfonamide compound [R. Soliman et al, J. Pharm. Sci., 70, 602 (1981)].

WO 00/27822 discloses tricyclic pyrazole derivatives, WO 00/59901 discloses dihydroindeno pyrazoles, WO 95/15315 discloses diphenyl pyrazole compounds, WO 95/15317 discloses triphenyl pyrazole compounds, WO 95/15318 discloses tri-substituted pyrazole compounds, and WO 96/09293 discloses benz[g]indazolyl derivatives. WO 95/15316 discloses substituted pyrazolyl benzenesulfamide derivatives.

### DETAILED DESCRIPTION OF THE INVENTION

A class of compounds, which are useful in treating cancer, inflammation, and inflammation related disorders, is defined by Formula I:

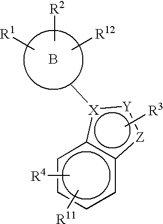

wherein

B is a 5 or 6 membered heteroaryl, aryl, saturated or unsaturated heterocyclic wherein said aryl, heteroaryl, or heterocyclic are optionally substituted with R¹, R², and R¹²;

X is selected from the group consisting of: N and C;

Y and Z are independently selected from the group consisting of: N, CH, CR³, S, and O;

R¹ is selected from the group consisting of: hydrido, halogen, alkyl, aryl, heteroaryl, alkenyl, alkynyl, haloalkyl, CN, NO₂, OR⁵, OCOOR⁵, C₂R⁷, CON(R⁶)R⁷, COR⁶, SR⁶, SOR⁶, SO₂R⁶, NR⁶R⁷, NR⁶COR⁷, NR⁶CONHR⁷, NR⁶SO₂R⁷, NR⁶SO₂NHR⁷, and SO₂N(R⁶)R⁷ wherein R⁶ and R⁷ may be taken together to form a 3-7 membered carbocyclic ring having 1 to 3 substituted or unsubstituted heteroatoms selected from the group consisting of: S, SO, SO₂, O, and NR⁶; wherein said alkenyl, alkynyl, alkyl, aryl, heteroaryl or OR⁵ are optional substituted with, hydrido, halogen, alkyl, hydroxyalkyl, aryl, heteroaryl, haloalkyl, COCF₃, CN, NO₂, OR⁵, OCOOR⁵, CO₂R⁷, CON(R⁶)R⁷, COR⁶, SR⁶, SOR⁶, SO₂R⁶, NR⁶R⁷, NR⁶COR⁷, NR⁶CONHR⁷, NR⁶SO₂R⁷, NR⁶SO₂NHR⁷, and SO₂N(R⁶)R⁷ wherein R⁶ and R⁷ may be taken together to form a 3-7 membered carbocyclic ring having 1 to 3 substituted or unsubstituted heteroatoms selected from the group consisting of: S, SO, SO₂, O, and NR⁶;

R² is selected from the group consisting of: halogen, hydrido, hydroxyalkyl, alkyl, OR⁶, CN, NO₂, SR⁶, NHR⁶, CON(R⁶)R⁷, NHCONHR⁶, CO₂H, and haloalkyl;

R¹ and R² may be taken together to form a 5 to 7 membered saturated or unsaturated carbocyclic ring optionally containing 0 to 3 heteroatoms selected from the group consisting of N, O, or S, and wherein said ring is optionally substituted with R¹;

R³ is selected from the group consisting of: substituted or unsubstituted amidine, alkylamino, aminoalkyl, CONHR⁷, NH₂, NHCOR⁶, and CH₂NHCOR⁶;

R⁴ is selected from the group consisting of: halogen, alkylsulfinyl, alkylsulfonyl, cyano, alkoxycarbonyl, alkyl, haloalkyl, hydrido, hydroxyalkyl, haloalkoxy, heterocyclic, nitro, acylamino, aryl, heteroaryl, and alkenyl, OR¹³, SR⁸, SO₂N(R⁸)R^{8′}, NHR⁹, NHCOR⁹, NR⁹COR⁹, NHCO(OR⁹), NR⁹CO(OR⁹), NR⁸SO₂R¹⁰, NHSO₂N(R¹⁰)R^{10′}, NR⁶CON(R¹⁰)R^{10′}, COR⁹, CO₂R⁸, CON(R⁸)R^{8′}, wherein R⁸ and R^{8′} may be taken together to form a 3-7 membered carbocyclic ring having 1 to 3 substituted or unsubstituted heteroatoms selected from S, SO, SO₂, O, N, and NR⁶, and wherein R¹⁰ and R^{10′} may be taken together to form a 3-7 membered carbocyclic ring having 1 to 3 substituted or unsubstituted heteroatoms selected from S, SO, SO₂, O, N, and NR⁶ wherein said aryl, heterocyclic, heteroaryl, or alkenyl are optionally substituted with R⁹;

R⁵ is selected from the group consisting of: hydrido, alkyl, aryl, arylalkyl, heteroaryl, heterocyclicalkyl, and heteroarylalkyl, wherein aryl, alkyl, arylalkyl, heteroaryl, heterocyclicalkyl, or heteroarylalkyl are optionally substituted with one or more radicals selected from the group consisting of OR¹⁴, N(R¹⁴)R^{14′}, and glycols;

R⁶ is independently selected from the group consisting of: hydrido, aryl, heteroaryl, lower alkyl, haloalkyl, alkenyl, alkynyl, hydroxyalkyl, aminoalkyl, alkylaminoalkyl, alkoxy, alkoxyalkyl, heterocyclicalkyl, and heterocyclic;

R⁷ is independently selected from the group consisting of: hydrido, aryl, heteroaryl, lower alkyl, haloalkyl, alkenyl, alkynyl, hydroxyalkyl, aminoalkyl, alkylaminoalkyl, alkoxy, alkoxyalkyl, heterocyclicalkyl, and heterocyclic;

R⁸ is independently selected from the group consisting of: hydrido, aryl, heteroaryl, arylalkyl, heterocyclic, haloalkyl, arylalkylamino, alkylaminoalkyl, dialkylaminoalkyl, alkyl, alkenyl, alkynyl, heteroarylalkyl, and heterocyclicalkyl;

R^{8′} is independently selected from the group consisting of: hydrido, aryl, heteroaryl, arylalkyl, heterocyclic, haloalkyl, arylalkylamino, alkylaminoalkyl, dialkylaminoalkyl, alkyl, alkenyl, alkynyl, heteroarylalkyl, and heterocyclicalkyl;

R⁹ is independently selected from the group consisting of: hydrido, lower alkyl, aryl, heteroaryl, arylalkyl, heterocyclic, cycloalkyl, heterocyclicalkyl, haloalkyl, arylalkylamino, amino, aminoalkyl, aminoacyl, nitro, azido, and heteroarylalkyl, wherein alkyl, aryl, heteroaryl, aminoalkyl, or arylalkyl are optionally substituted with one or more radical selected from the group consisting of: alkylsulfonamide, sulfamyl, alkyl, alkylthio, alkylsulfinyl, alkylsulfonyl, alkylamino, aminoalkyl, alkylaminoalkyl, alkoxy, halogen, acyloxy, oxy, formyl, haloalkyl, cyano, haloalkoxy, acyl, carboxyl, hydroxy, hydroxyalkyloxy, phenoxy, nitro, azido, benzyloxy, dialkylaminoacyl, thioalkyl, aminoacyloxy, thiocyanate, isothiocyanate, alkyldioxy, hydroxyalkyl, alkylamino, alkyloxycarbonyl, alkoxyalkyl, alkenylamino, alkynylamino, alkenyl, alkynyl, dialkylaminoalkyloxy, and heterocyclic optionally substituted with alkyl, alkylamino, aminoalkyl, and alkylaminoalkyl;

R¹⁰ is independently selected from the group consisting of: hydrido, lower alkyl, heteroaryl, heterocyclic, haloalkyl, arylalkylamino, heteroarylalkyl, aryl, and arylalkyl, wherein aryl, heteroaryl, heterocyclic, or arylalkyl are optionally substituted with one or more radical selected from alkyl, alkoxy, halogen, haloalkyl, cyano, haloalkoxy, acyl, carboxyl, hydroxy, hydroxyalkyloxy, phenoxy, benzyloxy, dialkylaminoalkyloxy, and heterocyclic,

R^{10′} is independently selected from the group consisting of: hydrido, lower alkyl, heteroaryl, heterocyclic, haloalkyl, arylalkylamino, heteroarylalkyl, aryl, and arylalkyl, wherein aryl, heteroaryl, heterocyclic, or arylalkyl are optionally substituted with one or more radical selected from alkyl, alkoxy, halogen, haloalkyl, cyano, haloalkoxy, acyl, carboxyl, hydroxy, hydroxyalkyloxy, phenoxy, benzyloxy, dialkylaminoalkyloxy, and heterocyclic,

R¹¹ is selected from the group consisting of: hydrido, halogen, haloalkyl, CN, CO₂R⁵, lower alkyl, lower alkenyl, lower alkynyl, alkoxy, and CONH₂;

R¹² is selected from the group consisting of: hydrido, halogen, alkyl, and alkoxy;

R¹³ is selected from the group consisting of: hydrido, alkyl, aryl, arylalkyl, heteroaryl, heterocyclicalkyl, and heteroarylalkyl, wherein aryl, alkyl, arylalkyl, heteroaryl, heterocyclicalkyl, or heteroarylalkyl are optionally substituted with one or more radicals selected from the group consisting of OR¹⁴, N(R¹⁴)R^{14′}, and glycols;

R¹⁴ is independently selected from the group consisting of hydrido, and lower alkyl; and

R^{14′} is independently selected from the group consisting of hydrido, and lower alkyl;

or isomers, tautomers, carriers, esters, prodrugs, pharmaceutically acceptable salts thereof.

Another class of compounds is defined by formula II

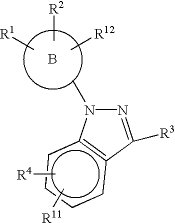

wherein

B is a 5 or 6 membered heteroaryl, aryl, saturated or unsaturated heterocyclic wherein said aryl, heteroaryl, or heterocyclic are optionally substituted with R¹, R², and R¹²;

R¹ is selected from the group consisting of: hydrido, halogen, alkyl, aryl, heteroaryl, alkenyl, alkynyl, haloalkyl, CN, NO₂, OR⁵, OCOOR⁵, CO₂R⁷, CON(R⁶)R⁷, COR⁶, SR⁶, SOR⁶, SO₂R⁶, NR⁶R⁷, NR⁶COR⁷, NR⁶CONHR⁷, NR⁶SO₂R⁷, NR⁶SO₂NHR⁷, and SO₂N(R⁶)R⁷ wherein R⁶ and R⁷ may be taken together to form a 3-7 membered carbocyclic ring having 1 to 3 substituted or unsubstituted heteroatoms selected from the group consisting of: S, SO, SO₂, O, and NR⁶; wherein said alkenyl, alkynyl, alkyl, aryl, heteroaryl or OR⁵ are optional substituted with, hydrido, halogen, alkyl, hydroxyalkyl, aryl, heteroaryl, haloalkyl, COCF₃, CN, NO₂, OR⁵, OCOOR⁵, CO₂R⁷, CON(R⁶)R⁷, COR⁶, SR⁶, SOR⁶, SO₂R⁶, NR⁶R⁷, NR⁶COR⁷, NR⁶CONHR⁷, NR⁶SO₂R⁷, NR⁶SO₂NHR⁷, and SO₂N(R⁶)R⁷ wherein R⁶ and R⁷ may be taken together to form a 3-7 membered carbocyclic ring having 1 to 3 substituted or unsubstituted heteroatoms selected from the group consisting of: S, SO, SO₂, O, and NR⁶;

R² is selected from the group consisting of: halogen, hydrido, hydroxyalkyl, alkyl, OR⁶, CN, NO₂, SR⁶, NHR⁶, CON(R⁶)R⁷, NHCONHR⁶, CO₂H, and haloalkyl;

R¹ and R² may be taken together to form a 5 to 7 membered saturated or unsaturated carbocyclic ring optionally containing 0 to 3 heteroatoms selected from the group consisting of N, O, or S, and wherein said ring is optionally substituted with R¹;

R³ is selected from the group consisting of: substituted or unsubstituted amidine, alkylamino, aminoalkyl, CONHR⁷, NH₂, NHCOR⁶, and CH₂NHCOR⁶;

R⁴ is selected from the group consisting of: halogen, alkylsulfinyl, alkylsulfonyl, cyano, alkoxycarbonyl, alkyl, haloalkyl, hydrido, hydroxyalkyl, haloalkoxy, heterocyclic, nitro, acylamino, aryl, heteroaryl, and alkenyl, OR¹³, SR⁸, SO₂N(R⁸)R^{8′}, NHR⁹, NHCOR⁹, NR⁹COR⁹, NHCO(OR⁹), NR⁹CO(OR⁹), NR⁸SO₂R¹⁰, NHSO₂N(R¹⁰)R^{10′}, NR⁶CON(R¹⁰)R^{10′}, COR⁹, CO₂R⁸, CON(R⁸)R^{8′}, wherein R⁸ and R^{8′} may be taken together to form a 3-7 membered carbocyclic ring having 1 to 3 substituted or unsubstituted heteroatoms selected from S, SO, SO₂, O, N, and NR⁶ and wherein R¹⁰ and R^{10′} may be taken together to form a 3-7 membered carbocyclic ring having 1 to 3 substituted or unsubstituted heteroatoms selected from S, SO, SO₂, O, N, and NR⁶ wherein said aryl, heterocyclic, heteroaryl, or alkenyl are optionally substituted with R⁹;

R⁵ is selected from the group consisting of: hydrido, alkyl, aryl, arylalkyl, heteroaryl, heterocyclicalkyl, and heteroarylalkyl, wherein aryl, alkyl, arylalkyl, heteroaryl, heterocyclicalkyl, or heteroarylalkyl are optionally substituted with one or more radicals selected from the group consisting of OR¹⁴, N(R¹⁴)R^{14′}, and glycols;

R⁶ is independently selected from the group consisting of: hydrido, aryl, heteroaryl, lower alkyl, haloalkyl, alkenyl, alkynyl, hydroxyalkyl, aminoalkyl, alkylaminoalkyl, alkoxy, alkoxyalkyl, heterocyclicalkyl, and heterocyclic;

R⁷ is independently selected from the group consisting of: hydrido, aryl, heteroaryl, lower alkyl, haloalkyl, alkenyl, alkynyl, hydroxyalkyl, aminoalkyl, alkylaminoalkyl, alkoxy, alkoxyalkyl, heterocyclicalkyl, and heterocyclic;

R⁸ is independently selected from the group consisting of: hydrido, aryl, heteroaryl, arylalkyl, heterocyclic, haloalkyl, arylalkylamino, alkylaminoalkyl, dialkylaminoalkyl, alkyl, alkenyl, alkynyl, heteroarylalkyl, and heterocyclicalkyl;

R^{8′} is independently selected from the group consisting of: hydrido, aryl, heteroaryl, arylalkyl, heterocyclic, haloalkyl, arylalkylamino, alkylaminoalkyl, dialkylaminoalkyl, alkyl, alkenyl, alkynyl, heteroarylalkyl, and heterocyclicalkyl;

R⁹ is independently selected from the group consisting of: hydrido, lower alkyl, aryl, heteroaryl, arylalkyl, heterocyclic, cycloalkyl, heterocyclicalkyl, haloalkyl, arylalkylamino, amino, aminoalkyl, aminoacyl, nitro, azido, and heteroarylalkyl, wherein alkyl, aryl, heteroaryl, aminoalkyl, or arylalkyl are optionally substituted with one or more radical selected from the group consisting of: alkylsulfonamide, sulfamyl, alkyl, alkylthio, alkylsulfinyl, alkylsulfonyl, alkylamino, aminoalkyl, alkylaminoalkyl, alkoxy, halogen, acyloxy, oxy, formyl, haloalkyl, cyano, haloalkoxy, acyl, carboxyl, hydroxy, hydroxyalkyloxy, phenoxy, nitro, azido, benzyloxy, dialkylaminoacyl, thioalkyl, aminoacyloxy, thiocyanate, isothiocyanate, alkyldioxy, hydroxyalkyl, alkylamino, alkyloxycarbonyl, alkoxyalkyl, alkenylamino, alkynylamino, alkenyl, alkynyl, dialkylaminoalkyloxy, and heterocyclic optionally substituted with alkyl, alkylamino, aminoalkyl, and alkylaminoalkyl;

R¹⁰ is independently selected from the group consisting of: hydrido, lower alkyl, heteroaryl, heterocyclic, haloalkyl, arylalkylamino, heteroarylalkyl, aryl, and arylalkyl, wherein aryl, heteroaryl, heterocyclic, or arylalkyl are optionally substituted with one or more radical selected from alkyl, alkoxy, halogen, haloalkyl, cyano, haloalkoxy, acyl, carboxyl, hydroxy, hydroxyalkyloxy, phenoxy, benzyloxy, dialkylaminoalkyloxy, and heterocyclic,

R^{10′} is independently selected from the group consisting of: hydrido, lower alkyl, heteroaryl, heterocyclic, haloalkyl, arylalkylamino, heteroarylalkyl, aryl, and arylalkyl, wherein aryl, heteroaryl, heterocyclic, or arylalkyl are optionally substituted with one or more radical selected from alkyl, alkoxy, halogen, haloalkyl, cyano, haloalkoxy, acyl, carboxyl, hydroxy, hydroxyalkyloxy, phenoxy, benzyloxy, dialkylaminoalkyloxy, and heterocyclic,

R¹¹ is selected from the group consisting of: hydrido, halogen, haloalkyl, CN, CO₂R⁵, lower alkyl, lower alkenyl, lower alkynyl, alkoxy, and CONH₂;

R¹² is selected from the group consisting of: hydrido, halogen, alkyl, and alkoxy;

R¹³ is selected from the group consisting of: hydrido, alkyl, aryl, arylalkyl, heteroaryl, heterocyclicalkyl, and heteroarylalkyl, wherein aryl, alkyl, arylalkyl, heteroaryl, heterocyclicalkyl, or heteroarylalkyl are optionally substituted with one or more radicals selected from the group consisting of OR¹⁴, N(R¹⁴)R^{14′}, and glycols;

R¹⁴ is independently selected from the group consisting of hydrido, and lower alkyl; and

R^{14′} is independently selected from the group consisting of hydrido, and lower alkyl;

or isomers, tautomers, carriers, esters, prodrugs, pharmaceutically acceptable salts thereof.

Definitions

The present invention includes the use of all hydrates, solvates, complexes and prodrugs of the compounds of this invention. Prodrugs are any covalently bonded compounds, which releases the active parent drug according to Formula I or Formula II in vivo. If a chiral center or another form of an isomeric center is present in a compound of the present invention all forms of such isomer or isomers, including enantiomers and diastereomers, are intended to be covered herein. Compounds containing a chiral center may be used as a racemic mixture, an enantiornerically enriched mixture, or the racemic mixture may be separated using well-known techniques and an individual enantiomer may be used alone. In cases in which compounds have unsaturated carbon-carbon double bonds, both the cis (Z) and trans (E) isomers are within the scope of this invention. In cases wherein compounds may exist in tautomeric forms, such as keto-enol tautomers, each tautomeric form is contemplated as being included within this invention whether existing in equilibrium or predominantly in one form.

The meaning of any substituent at any one occurrence in Formula I or Formula II or any sub-formula thereof is independent of its meaning, or any other substituents meaning, at any other occurrence, unless specified otherwise.

The term “alkyl” is used, either alone or within other terms such as “haloalkyl” and “alkylsulfonyl”; it embraces linear or branched radicals having one to about twenty carbon atoms or, preferably, one to about twelve carbon atoms. More preferred alkyl radicals are “lower alkyl” radicals having one to about ten carbon atoms. Most preferred are lower alkyl radicals having one to about five carbon atoms. Examples of such radicals include methyl, ethyl, n-propyl, isopropyl, n-butyl, isobutyl, sec-butyl, tert-butyl, pentyl, isoamyl, hexyl, octyl and the, like. The term “hydrido” denotes a single hydrogen atom (H). This hydrido radical may be attached, for example, to an oxygen atom to form a hydroxyl radical or two hydrido radicals may be attached to a carbon atom to form a methylene (—CH2-) radical. The term “halo” means halogens such as fluorine, chlorine, and bromine or iodine atoms. The term “haloalkyl” embraces radicals wherein any one or more of the alkyl carbon atoms is substituted with halo as defined above. Specifically embraced are monohaloalkyl, dihaloalkyl, and polyhaloalkyl radicals. A monohaloalkyl radical, for one example, may have a bromo, chloro, or a fluoro atom within the radical. Dihalo radicals may have two or more of the same halo atoms or a combination of different halo radicals and polyhaloalkyl radicals may have more than two of the same halo atoms or a combination of different halo radicals. The term “hydroxyalkyl” embraces linear or branched alkyl radicals having one to about ten carbon atoms any one of which may be substituted with one or more hydroxylradicals. The terms “alkoxy” and “alkoxyalkyl” embrace linear or branched oxy-containing radicals each having alkyl portions of one to about ten carbon atoms, such as methoxy radical. The term “alkoxyalkyl” also embraces alkyl radicals having two or more alkoxy radicals attached to the alkyl radical, that is, to form monoalkoxyalkyl and dialkoxyalkyl radicals. The “alkoxy” or “alkoxyalkyl” radicals may be further substituted with one or more halo atoms, such as fluoro, chloro, or bromo, to provide “haloalkoxy” or “haloalkoxyalkyl” radicals. Examples of “alkoxy” radicals include methoxy, butoxy, and trifluoromethoxy. The term “aryl”, alone or in combination, means a carbocyclic aromatic system containing one, two, or three rings wherein such rings may be attached together in a pendent manner or may be fused. The term “aryl” embraces aromatic radicals such as phenyl, naphthyl, tetrahydronapthyl, indane, and biphenyl. The term “heterocyclic” embraces saturated, partially saturated, and unsaturated heteroatom-containing ring-shaped radicals, where the heteroatoms may be selected from nitrogen, sulfur and oxygen. Examples of saturated heterocyclic radicals include pyrrolidyl and morpholinyl. The term “heteroaryl” embraces unsaturated heterocyclic radicals. Examples of unsaturated heterocyclic radicals, also termed “heteroaryl” radicals include thienyl, pyrrolyl, furyl, pyridyl, pyrimidyl, pyrazinyl, pyrazolyl, oxazolyl, isoxazolyl, imidazolyl, thiazolyl, and tetrazolyl. The term also embraces radicals where heterocyclic radicals are fused with aryl radicals. Examples of such fused bicyclic radicals include benzofuran, benzothiophene, and the like. The term “heterocyclic alkyl” embraces alkyl attached to the heterocyclic. The term “sulfonyl”, whether used alone or linked to other terms such as alkylsulfonyl, denotes respectively divalent radicals —SO₂—. “Alkylsulfonyl”, embraces alkyl radicals attached to a sulfonyl radical, where alkyl is defined as above. The term “arylsulfonyl” embraces sulfonyl radicals substituted with an aryl radical. The terms “sulfamyl” or “sulfonamidyl”, whether alone or used with terms such as “N-alkylsulfamyl”, “N-arylsulfamyl”, “N,N-dialkylsulfamyl” and “N-alkyl-N-arylsulfamyl”, denotes a sulfonyl radical substituted with an amine radical, forming a sulfonamide (—SO₂—NH₂). The terms “N-alkylsulfamyl” and “N,N-dialkylsulfamyl” denote sulfamyl radicals substituted, respectively, with one alkyl radical, a cycloalkyl ring, or two alkyl radicals. The terms “N-arylsulfamyl” and “N-alkyl-N-arylsulfamyl” denote sulfamyl radicals substituted, respectively, with one aryl radical, and one alkyl and one aryl radical. The terms “carboxy” or “carboxyl”, whether used alone or with other terms, such as “carboxyalkyl”, denotes —CO₂H. The term “carboxyalkyl” embraces radicals having a carboxyradical as defined above, attached to an alkyl radical. The term “carbonyl”, whether used alone or with other terms, such as “alkylcarbonyl”, denotes —(C═O)—. The term “alkylcarbonyl” embraces radicals having a carbonyl radical substituted with an alkyl radical. An example of an “alkylcarbonyl” radical is CH₃—(C═O)—. The term “alkylcarbonylalkyl” denotes an alkyl radical substituted with an “alkylcarbonyl” radical. The term “alkoxycarbonyl” means a radical containing an alkoxy radical, as defined above, attached via an oxygen atom to a carbonyl (C═O) radical. Examples of such “alkoxycarbonyl” radicals include (CH₃)₃CO—C═O)— and —(O═)C—OCH₃. The term “alkoxycarbonylalkyl” embraces radicals having “alkoxycarbonyl”, as defined above substituted to an alkyl radical. Examples of such “alkoxycarbonylalkyl” radicals include (CH₃)₃COC(═O) (CH₂)₂— and —(CH₂)₂(O═)COCH₃. The term “amido” when used by itself or with other terms such as “amidoalkyl”, “N-monoalkylamido”, “N-monoarylamido”, “N,N-dialkylamido”, “N-alkyl-N-arylamido”, “N-alkyl-N-hydroxyamido” and “N-alkyl-N-hydroxyamidoalkyl”, embraces a carbonyl radical substituted with an amino radical. The terms “N-alkylamido” and “N,N-dialkylamido” denote amido groups which have been substituted with one alkyl radical and with two alkyl radicals, respectively. The terms “N-monoarylamido” and “N-alkyl-N-arylamido” denote amido radicals substituted, respectively, with one aryl radical, and one alkyl and one aryl radical. The term “N-alkyl-N-hydroxyamido” embraces amido radicals substituted with a hydroxyl radical and with an alkyl radical. The term “N-alkyl-N-hydroxyamidoalkyl” embraces alkyl radicals substituted with an N-alkyl-N-hydroxyamido radical. The term “amidoalkyl” embraces alkyl radicals substituted with amido radicals. The term “aminoalkyl” embraces alkyl radicals substituted with amino radicals. The term “alkylaminoalkyl” embraces aminoalkyl radicals having the nitrogen atom substituted with an alkyl radical. The term “amidino” denotes an —C(═NH)—NH₂ radical. The term “cyanoamidino” denotes an —C(═N—CN)—NH₂ radical. The term “heterocycloalkyl” embraces heterocyclic-substituted alkyl radicals such as pyridylmethyl and thienylmethyl. The term “aralkyl” embraces aryl-substituted alkyl radicals such as benzyl, diphenylmethyl, triphenylmethyl, phenethyl, and diphenethyl. The terms benzyl and phenylmethyl are interchangeable. The term “cycloalkyl” embraces radicals having three to ten carbon atoms, such as cyclopropyl cyclobutyl, cyclopentyl, cyclohexyl, and cycloheptyl. The term “cycloalkenyl” embraces unsaturated radicals having three to ten carbon atoms, such as cylopropenyl, cyclobutenyl, cyclopentenyl, cyclohexenyl, and cycloheptenyl. The term “alkylthio” embraces radicals containing a linear or branched alkyl radical, of one to ten carbon atoms, attached to a divalent sulfur atom. An example of “alkylthio” is methylthio, (CH₃—S—). The term “alkylsulfinyl” embraces radicals containing a linear or branched alkyl radical, of one to ten carbon atoms, attached to a divalent —S(═)— atom. The terms “N-alkylamino” and “N,N-dialkylamino” denote amino groups which have been substituted with one alkyl radical and with two alkyl radicals, respectively. The term “acyl”, whether used alone, or within a term such as “acylamino”, denotes a radical provided by the residue after removal of hydroxyl from an organic acid. The term “acylamino” embraces an amino radical substituted with an acyl group. An examples of an “acylamino” radical is acetylamino (CH₃C(═O)—NH—).

Compounds of Formula I or Formula II would be useful for, but not limited to, the treatment of inflammation in a subject, and for treatment of other inflammation-associated disorders, such as, as an analgesic in the treatment of pain and headaches, or as an antipyretic for the treatment of fever. For example, compounds of Formula I or Formula II would be useful to treat arthritis, including but not limited to rheumatoid arthritis, spondylo arthopathies, gouty arthritis, osteoarthritis, systemic lupus erythematosus, and juvenile arthritis. Such compounds of Formula I or Formula II would be useful in the treatment of asthma, bronchitis, menstrual cramps, tendinitis, bursitis, and skin related conditions such as psoriasis, eczema, burns, and dermatitis. Compounds of Formula I or Formula II also would be useful to treat gastrointestinal conditions such as inflammatory bowel disease, Crohn's disease, gastritis, irritable bowel syndrome, and ulcerative colitis and for the prevention of colorectal cancer. Compounds of Formula I or Formula II would be useful in treating inflammation in such diseases as vascular diseases such as vascularitus, migraine headaches, periarteritis nodosa, thyroiditis, aplastic anemia, Hodgkin's disease, sclerodoma, rheumatic fever, type I diabetes, myasthenia gravis, sarcoidosis, nephrotic syndrome, Behcet's syndrome, polymyositis, gingivitis, hypersensitivity, conjunctivitis, swelling occurring after injury, myocardial ischemia, and the like. The compounds of the present invention may also be used for pain. The compounds are useful as antiinflammatory agents, such as for the treatment of arthritis, with the additional benefit of having significantly less harmful side effects. The compounds of formula I or II are useful as agents for treating cancer or anticancer agents. The compounds of formula I or II may be proapoptotic, antiapoptotic, anticell cycle progressive, antiinvasive, antiproliferative, antiangiogenic, and antimetastatic. The cancer may be colon, ovarian, breast, prostate, gastric, B-cell lymphoma, and multiple myeloma. More specifically, the compounds of this invention are useful in the treatment of a variety of cancers including, but not limited to: carcinoma such as bladder, breast, colon, kidney, liver, lung, including small cell lung cancer, esophagus, gall-bladder, ovary, pancreas, stomach, cervix, thyroid, prostate, and skin, including squamous cell carcinoma; hematopoietic tumors of lymphoid lineage, including leukemia, acute lymphocytic leukemia, acute lymphoblastic leukemia, B-cell lymphoma, T-cell-lymphoma, Hodgkin's lymphoma, non-Hodgkin's lymphoma, hairy cell lymphoma and Burkett's lymphoma; hematopoietic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including astrocytoma, neuroblastoma, glioma and schwannomas; other tumors, including melanoma, seminoma, teratocarcinoma, osteosarcoma, xeroderma pigmentosum, keratoxanthoma, thyroid follicular cancer and Kaposi's sarcoma. Due to the key role of PKs in the regulation of cellular proliferation, these compounds are also useful in the treatment of a variety of cell proliferative disorders such as, for instance, benign prostate hyperplasia, familial adenomatosis, polyposis, neuro-fibromatosis, psoriasis, vascular smooth cell proliferation associated with atherosclerosis, pulmonary fibrosis, arthritis glomerulonephritis and post-surgical stenosis and restenosis. The compounds of formula I or II may be used as an anitviral agent. The compounds of this invention are useful as inhibitors of protein kinases. The compounds of this invention are useful as inhibitors of IKK1 and/or IKK2, IKKα/IKKβ heterodimer, TBK or IKKi. The compounds of the invention may also useful as inhibitors of other protein kinases such as, for instance, protein kinase C in different isoforms, cyclin dependent kinase (cdk), Met, PAK-4, PAK-5, ZC-1, STLK-2, DDR-2, Aurora 1, Aurora 2, Bub-1, PLK, Chk1, Chk2, HER2, raf1, MEK1, MAPK, EGF-R, PDGF-R, FGF-R, IGF-R, VEGF-R, PI3K, weel kinase, Src, Ab1, Akt, ILK, MK-2, IKK-2, Cdc7, Nek, and thus be effective in the treatment of diseases associated with other protein kinases. The present invention preferably includes compounds, which selectively inhibit IKK2 over IKK1. Preferably, the compounds have an IKK2 IC50 of less than 1 μM, and have a selectivity ratio of IKK2 inhibition over IKK1 inhibition of at least 50, and more preferably of at least 100. Even more preferably, the compounds have an IKK1 IC50 of greater than 10 μM, and more preferably of greater than 100 μM. The compounds of formula may also be used to treat angiogenesis associated cardiovascular, ophthalmology and osteoporosis disorders. The compounds of the present invention may also be used for treatment of knee injury such as sport injuries.

While it is possible for an active ingredient to be administered alone as the raw chemical, it is preferable to present it as a pharmaceutical formulation. The present invention comprises a pharmaceutical composition comprising a therapeutically effective amount of a compound of the present invention in association with at least one pharmaceutically acceptable carrier, adjuvant, or diluent. The present invention also comprises a method of treating inflammation or inflammation associated disorders in a subject, the method comprising administering to the subject having such inflammation or disorders a therapeutically effective amount of a compound of the present invention. Also included in the family of compounds of the present invention are the pharmaceutically acceptable salts thereof. The term “pharmaceutically acceptable salts” embraces salts commonly used to form alkali metal salts and to form addition salts of free acids or free bases. The nature of the salt is not critical, provided that it is pharmaceutically acceptable. Suitable pharmaceutically acceptable acid addition salts of compounds of the present invention may be prepared from an inorganic acid or from an organic acid. Examples of such inorganic acids are hydrochloric, hydrobromic, hydroiodic, nitric, carbonic, sulfuric, and phosphoric acid. Appropriate organic acids may be selected from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, salicyclic, salicyclic, phydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, stearic, cyclohexylaminosulfonic, algenic, β-hydroxybutyric, salicyclic, galactaric and galacturonic acid. Suitable pharmaceutically acceptable base addition salts of compounds of the present invention include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine (N-methyl-glucamine) and procaine. All of these salts may be prepared by conventional means from the corresponding compound of the present invention by reacting, for example, the appropriate acid or base with the compound of the present invention.

Also embraced within this invention are pharmaceutical compositions comprising one or more compounds of the present invention in association with one or more non-toxic, pharmaceutically acceptable carriers and/or diluents and/or adjuvants and/or excipient (collectively referred to herein as “carrier” materials) and, if desired, other active ingredients. Accordingly, the compounds of the present invention may be used in the manufacture of a medicament. Pharmaceutical compositions of the compounds of the present invention prepared as herein before described may be formulated as solutions or lyophilized powders for parenteral administration. Powders may be reconstituted by addition of a suitable diluent or other pharmaceutically acceptable carrier prior to use. The liquid formulation may be a buffered, isotonic aqueous solution. The compounds of the present invention may be administered by any suitable route, preferably in the form of a pharmaceutical composition adapted to such a route, and in a dose effective for the treatment intended. The compounds and composition may, for example, be administered intravascularly, intraperitoneally, intravenously, subcutaneously, intramuscularly, intramedullary, orally, or topically. For oral administration, the pharmaceutical composition may be in the form of, for example, a tablet, capsule, suspension, or liquid. The active ingredient may also be administered by injection as a composition wherein, for example, normal isotonic saline solution, standard 5% dextrose in water or buffered sodium or ammonium acetate solution may be used as a suitable carrier. Such formulation is especially suitable for parenteral administration, but may also be used for oral administration or contained in a metered dose inhaler or nebulizer for insufflation. It may be desirable to add excipients such as polyvinylpyrrolidone, gelatin, hydroxy cellulose, acacia, polyethylene glycol, mannitol, sodium chloride, or sodium citrate. The pharmaceutical composition is preferably made in the form of a dosage unit containing a particular amount of the active ingredient. Examples of such dosage units are tablets or capsules. The amount of therapeutically active compound that is administered and the dosage regimen for treating a disease condition with the compounds and/or compositions of this invention depends on a variety of factors, including the age, weight, sex and medical condition of the subject, the severity of the disease, the route and frequency of administration, and the particular compound employed, and thus may vary widely. The pharmaceutical compositions may contain active ingredient in the range of about 0.1 to 2000 mg, preferably in the range of about 0.5 to 500 mg and most preferably between about 1 and 100 mg. A daily dose of about 0.01 to 100 mg/kg bodyweight, preferably between about 0.1 and about 50 mg/kg body weight and most preferably between about 1 to 20 mg/kg bodyweight, may be appropriate. The daily dose can be administered in one to four doses per day. For therapeutic purposes, the compounds of this invention are ordinarily combined with one or more adjuvants appropriate to the indicated route of administration. If administered orally, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled release formulation as may be provided in a dispersion of active compound in a sustained release material such as glyceryl monostearate, glyceryl distearate, hydroxypropylmethyl cellulose alone or with a wax. Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, and/or various buffers. The pharmaceutical preparations are made following the conventional techniques of pharmacy involving milling, mixing, granulating, and compressing, when necessary, for tablet forms; or milling, mixing and filling for hard gelatin capsule forms. When a liquid carrier is used, the preparation will be in the form of a syrup, elixir, emulsion, or an aqueous or non-aqueous suspension. Such a liquid formulation may be administered orally or filled into a soft gelatin capsule. For rectal administration, the compounds of the present invention may also be combined with excipients such as cocoa butter, glycerin, gelatin, or polyethylene glycols and molded into a suppository. The methods of the present invention include topical administration of the compounds of the present invention. By topical administration is meant non-systemic administration, including the application of a compound of the invention externally to the epidermis, to the buccal cavity and instillation of such a compound into the ear, eye, and nose, wherein the compound does not significantly enter the blood stream. By systemic administration is meant oral, intravenous, intraperitoneal, and intramuscular administration. The amount of a compound of the present invention (hereinafter referred to as the active ingredient) required for therapeutic or prophylactic effect upon topical administration will, of course, vary with the compound chosen, the nature and severity of the condition being treated and the animal undergoing treatment, and is ultimately at the discretion of the physician.

The topical formulations of the present invention, both for veterinary and for human medical use, comprise an active ingredient together with one or more acceptable carriers therefore, and optionally any other therapeutic ingredients. The carrier must be “acceptable” in the sense of being compatible with the other ingredients of the formulation and not deleterious to the recipient thereof. Formulations suitable for topical administration include liquid or semi-liquid preparations suitable for penetration through the skin to the site of where treatment is required such as: liniments, lotions, creams, ointments or pastes, and drops suitable for administration to the eye, ear or nose. The active ingredient may comprise, for topical administration, from 0.01 to 5.0 wt %. of the formulation.

Drops according to the present invention may comprise sterile aqueous or oily solutions or suspensions and may be prepared by dissolving the active ingredient in a suitable aqueous solution of a bactericidal and/or fungicidal agent and/or any other suitable preservative, and preferably including a surface active agent. The resulting solution may then be clarified by filtration, transferred to a suitable container, which is then sealed and sterilized by autoclaving, or maintaining at 90-100° C. for half an hour. Alternatively, the solution may be sterilized by filtration and transferred to the container by an aseptic technique. Examples of bactericidal and fungicidal agents suitable for inclusion in the drops are phenylmercuric nitrate or acetate (0.00217c), benzalkonium chloride (0.0 1%) and chlorhexidine acetate (0.0 1%). Suitable solvents for the preparation of an oily solution include glycerol, diluted alcohol, and propylene glycol.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil. Creams, ointments, or pastes according to the present invention are semi-solid formulations of the active ingredient for external application. They may be made by mixing the active ingredient in finely divided or powdered form, alone or in solution or suspension in an aqueous or non-aqueous fluid, with the aid of suitable machinery, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol or macrogols. The formulation may incorporate any suitable surface-active agent such as an anionic, cationic, or non-ionic surface-active agent such as sorbitan esters or polyoxyethylene derivatives thereof. Suspending agents such as natural gums, cellulose derivatives or inorganic materials such as silicaceous silicas, and other ingredients such as lanolin may also be included. Other adjuvants and modes of administration are well and widely known in the pharmaceutical art. Although this invention has been described with respect to specific embodiments, the details of these embodiments are not to be construed as limitations.

Another aspect of the present invention is chemical intermediates in the synthesis of the claimed compounds.

Another aspect of the present invention is methods of syntheses of the claimed compounds.

### General Synthetic Procedures

The starting materials used herein are commercially available or are prepared by routine methods well known to those of ordinary skill in the art and can be found in standard reference books, such as the COMPENDIUM OF ORGANIC SYNTHETIC METHODS, Vol. I-VI (published by Wiley-Interscience)

The compounds of the invention can be synthesized according to the following procedures of Schemes I and II, wherein the R1-R11 substituents, are as defined for Formula I or II, above, except where further noted.

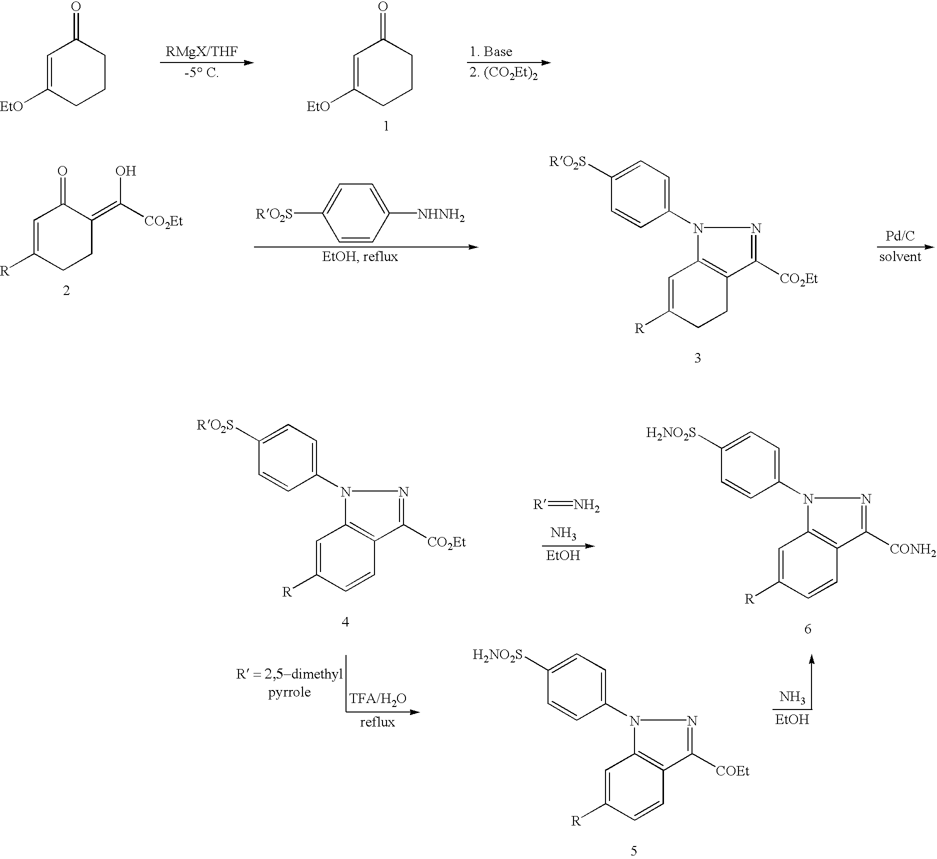

Scheme I shows the general synthesis of 6-substitutedindazole. The commercially available 3-ethoxy-2-cyclohexen-1-one is reacted with Grignard reagents such as substituted aryl, pyridyl magnesium bromides to give ketone 1. This ketone is first treated with a base, then reacted with diethyl oxylate to afford 1,3-diketone 2 which exists predominantly in ketol form. Examples of suitable base are lithium hexamethyldisilazide, sodium ethoxide. The resulting 1,3-diketone 2 is then condensed with hydrazine to give pyrazole 3. Examples of suitable hydrazines are 4-sulfonamidophenylhydrazine, 4-methylsulfonylphenylhydrazine and 1-(4-hydrazinophenylsulfonyl)-2,5-dimethylpyrrole. The conversion of pyrazole 3 to indazole 4 is accomplished by aromatization catalyzed by 10% Pd/C in a suitable solvent such as xylene or cumene. The indazole 4 is then converted to amide 6 by treatment with liquid ammonia in ethanol in a sealed vessel. In the case where the sulfonamide nitrogen was protected as a 2,5-dimethylpyrrole, the deprotection was carried out by refluxing in TFA/water media to give 5, followed by amidation.

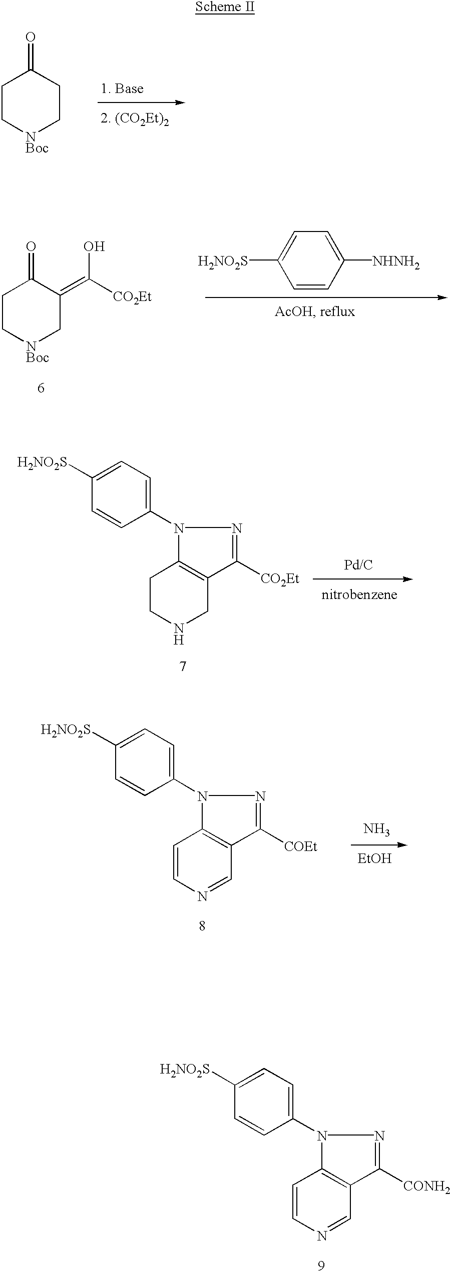

Scheme II shows the 4-step synthesis of pyrazolo[4,3-c]pyridine. In step 1, the commercially available N-Bocpiperidone was treated with a base, then reacted with diethyl oxylate to afford 1,3-diketone 6. Examples of suitable base are lithium hexamethyldisilazide, sodium ethoxide. In step 2, the resulting 1,3-diketone 6 is condensed with 4-sulfonamidophenylhydrazine to give pyrazole 7. The pyrazole 7 is then dehydrogenated with 10% Pd/C in nitrobenzene to give pyrazolo[4,3-c]pyridine 8 in step 3. Finally, the conversion of 8 to amide 9 is accomplished by treatment with liquid ammonia in ethanol in a sealed vessel.

The complete content of all publications, patents, and patent applications cited in this disclosure are herein incorporated by reference as if each individual publication, patent, or patent application were specifically and individually indicated to incorporated by reference. Although the foregoing invention has been described in some detail by way of illustration and example for the purposes of clarity of understanding, it will be readily apparent to one skilled in the art in light of the teachings of this invention that changes and modifications can be made without departing from the spirit and scope of the present invention. The following examples are provided for exemplification purposes only and are not intended to limit the scope of the invention, which has been described in broad terms above.

### EXAMPLES

### Example 1

### 1-[4-(aminosulfonyl)phenyl]-6-(4-methoxyphenyl)-1H-indazole-3-carboxamide

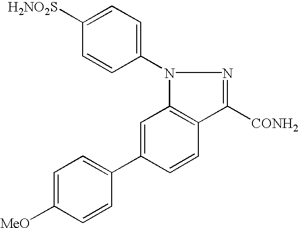

Step 1

To a solution of 4-methoxyphenyl magnesium bromide (100 mL, 0.5 M in THF) was added a solution of 3-ethoxy-2-cyclohexe-1-none (7.1 g, 0.05 mol) in 25 mL of dry THF at −5° C. over 15 minutes. The reaction mixture was stirred at −5˜0° C. for 0.5 h and room temperature for 2 h. The brown solution was poured into 400 mL of 1.5 N HCl and stirred for 1 h. The aqueous phase was then extracted with ethyl acetate (2×200 mL). The combined organic layers were washed with brine, dried over magnesium sulfate, and filtered. The filtrate was concentrated to give 10.2 g of crude as a yellow semisolid that was used without further purification in the next step.

Step 2

To a solution of lithium bis(trimethylsilyl)amide (17 mL, 1.0 M in THF) in 20 mL of dry ether at −78° C. was added a solution of the crude from step 1 (3.3 g, 0.016 mol) in 20 mL of ether slowly. The reaction mixture was stirred at this temperature for 0.5 h. Then a solution of diethyl oxylate (2.5 g, 0.017 mol) in 10 mL of dry ether was added in one portion. The mixture was stirred overnight while warming up to room temperature. Water (200 mL) was added and the aqueous phase was neutralized with 1 N HCl, extracted with ethyl acetate. The organic layer was washed with brine, dried over magnesium sulfate, and filtered. The filtrate was concentrated to give 5.13 g of crude as a brown solid that was used without further purification in the next step.

Step 3

A mixture of the crude from step 2 (3.1 g, 0.01 mol) and 4-sulfonamidophenylhydrazine hydrochloride (2.5 g, 0.01 mol) in 50 mL of absolute alcohol was heated at reflux overnight. After cooling, the suspension was filtered to give 3.8 g of product as a yellow solid. The mother liquor was concentrated and triturated with ether to give another 0.7 g of pure product; mp: 230-231° C.; Anal. Calcd. for C₂₃H₂₂N₃O₅S: C, 61.05; H, 4.90; N, 9.29; S, 7.09. Found: C, 60.84; H, 5.19; N, 9.62; S, 7.40.

Step 4

A mixture of the product from step 3 (2.3 g, 0.005 mol) and 1.2 g of 10% Pd/C in 100 mL of cumene was stirred at reflux overnight. After cooling, the mixture was filtered through a pad of Celite® and the filtrate was concentrated. The crude was recrystallized from methanol to give 1.1 g of product as a white solid; mp: 134-136° C.; Anal. Calcd. for C₂₃H₂₀N₃O₅S: C, 61.32; H, 4.47; N, 9.33; S, 7.12. Found: C, 61.84; H, 5.02; N, 8.81; S, 6.93.

Step 5

A sealed reaction vessel containing the product from step 4 (0.85 g, 0.0019 mol) and 20 mL of liquid ammonia in 100 mL of absolute alcohol was heated at 90° C. and 250 PSI for 20 h. After cooling, the precipitate was filtered and air-dried to give 0.54 g of product as a white crystal; mp: 258-259° C.; Anal. Calcd. for C₂₁H₁₈N₄O₄S: C, 57.14; H, 4.11; N, 12.69; S, 7.26. Found: C, 57.11; H, 4.15; N, 12.63; S, 7.25.

### Example 2

### 1-[4-(aminosulfonyl)phenyl]-6-(4-fluorophenyl)-1H-indazole-3-carboxamide

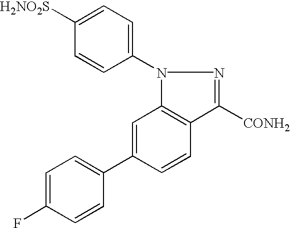

This compound was synthesized by using the same method described in Example 1 except using 4-fluorophenylmagnesium bromide in Step 1; mp: 314-315° C.; Anal. Calcd. for C₂₀H₁₅FN₄O₃S: C, 58.53; H, 3.68; N, 13.65; S, 7.81. Found: C, 58.12; H, 4.14; N, 13.06; S, 7.15.

### Example 3

### 1-[4-(aminosulfonyl)phenyl]-6-(3-methylphenyl)-1H-indazole-3-carboxamide

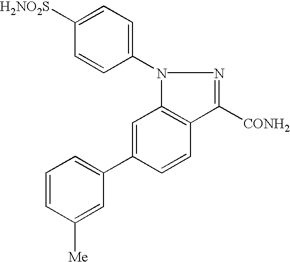

This compound was synthesized by using the same method described in Example 1 except using 3-methylphenylmagnesium bromide in Step 1; mp: 261-263° C.; Anal. Calcd. for C₂₁H₁₈N₄O₃S: C, 62.05; H, 4.46; N, 13.78; S, 7.89. Found: C, 61.72; H, 4.41; N, 13.72; S, 8.03.

### Example 4

### 1-[4-(aminosulfonyl)phenyl]-6-(4-tert-butylphenyl)-1H-indazole-3-carboxamide

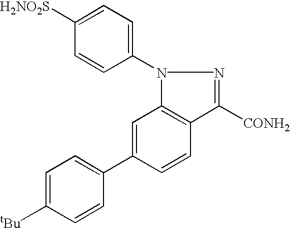

This compound was synthesized by using the same method described in Example 1 except using 4-tert-butylphenylmagnesium bromide in Step 1; mp: 262-263° C.; Anal. Calcd. for C₂₄H₂₄N₄O₃S: C, 64.27; H, 5.39; N, 12.49; S, 7.15. Found: C, 63.93; H, 5.42; N, 12.23; S, 7.23.

### Example 5

### 1-[4-(aminosulfonyl)phenyl]-6-(4-fluoro-3-methylphenyl)-1H-indazole-3-carboxamide

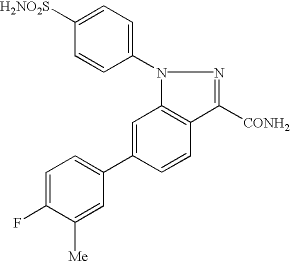

This compound was synthesized by using the same method described in Example 1 except using 4-fluoro-3-methylphenylmagnesium bromide in Step 1; mp: 300-302° C.; Anal. Calcd. for C₂₁H₁₇FN₄O₃S: C, 59.42; H, 4.04; N, 13.20; S, 7.55. Found: C, 59.03; H, 3.98; N, 12.92; S, 7.49.

### Example 6

### 1-[4-(aminosulfonyl)phenyl]-6-[3-(dimethylamino)phenyl]-1H-indazole-3-carboxamide

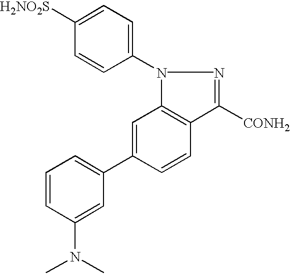

Step 1

To a mixture of 3-bromoaniline (84 g, 0.48 mol) in 100 mL of water at 0° C. was added dimethyl sulfate (60.9 g, 0.48 mol) dropwise. The reaction mixture was stirred for 1 h and then neutralized with 25% NaOH. Another equivalent of dimethyl sulfate was added and stirring was continued for 1 h. After adjusting pH to 8, half equivalent of dimethyl sulfate was added. The mixture was stirred for 1 h and then was basified. The aqueous phase was extracted with ether and combined organic layers were washed with brine, dried over magnesium sulfate, and filtered. The filtrate was concentrated and purified by vacuum distillation to give 53 g of 3-bromo-N,N-dimethylaniline as a clear liquid (107° C./25 mmHg). To a solution of 3-bromo-N,N-dimethylaniline (10.0 g, 0.05 mol) and magnesium (1.2 g, 0.05 mol) in dry THF was added a catalytical amount of iodine. The reaction mixture was heated at reflux for 2 h and then cooled to −5° C. To this was added a solution of 3-ethoxy-2-cyclohexe-1-none (7.1 g, 0.05 mol) in 25 mL of dry THF at −5° C. over 15 minutes. The reaction mixture was stirred at −5˜0° C. for 0.5 h and room temperature for 12 h. The brown solution was poured into 400 mL of 1.5 N HCl and stirred for 1 h. The aqueous phase was then extracted with ethyl acetate (2×200 mL). The combined organic layers were washed with brine, dried over magnesium sulfate, and filtered. The filtrate was concentrated to give 8.5 g of crude as a yellow oil, which was used without further purification in the next step.

Step 2

To a solution of lithium bis(trimethylsilyl)amide (40 mL of 1.0 M in THF) in 20 mL of dry ether at −78° C. was added a solution of the crude from step 1 (8.4 g, 0.039 mol) in 40 mL of ether slowly. The reaction mixture was stirred at this temperature for 0.5 h. Then a solution of diethyl oxylate (5.8 g, 0.039 mol) in 20 mL of dry ether was added in one portion. The mixture was stirred overnight while warming up to room temperature. Water (200 mL) was added and the aqueous phase was neutralized with 1 N HCl, extracted with ethyl acetate. The organic layer was washed with brine, dried over magnesium sulfate, and filtered. The filtrate was concentrated to give 11.9 g of crude as a brown solid, which was used without further purification in the next step.

Step 3

A mixture of the crude from step 2 (8.9 g, 0.028 mol) and 1-(4-hydrazinophenylsulfonyl)-2,5-dimethylpyrrole (7.95 g, 0.03 mol) in 100 mL of acetic acid was heated at reflux for 3 h. After cooling, the suspension was diluted with ethyl acetate and filtered to afford 14.5 g of product as a yellow solid (95% yield); mp: 176-178° C.; Anal. Calcd. for C₃₀H₃₂N₄O₄S: C, 66.16; H, 5.92; N, 10.29; S, 5.89. Found: C, 65.50; H, 5.83; N, 10.06; S, 5.97.

Step 4

A mixture of the product from step 3 (14.0 g, 0.026 mol) and 6.2 g of 10% Pd/C in 350 mL of cumene and 20 mL of N-methylpyrrolidone was stirred at reflux for 3 days. After cooling, the mixture was filtered through a pad of Celite® and the filtrate was concentrated. The crude was purified by chromatography on silica gel (ethyl acetate/hexane, 3:7) to give 3.1 g of product as a yellow solid; mp: 160-161° C. Anal. Calcd. for C₂₉H₂₈N₄O₄S: C, 66.40; H, 5.57; N, 10.32; S, 5.91. Found: C, 66.41; H, 5.49; N, 10.06; S, 5.87.

Step 5

A solution of the product from step 4 (0.7 g, 0.0012 mol) in a mixture of TFA (15 mL) and water (5 mL) was heated at reflux for 2.5 h. The solvent was removed and the residue was basified with ammonium hydroxide solution and extracted with methylene chloride. The organic layer was washed with brine, dried over magnesium sulfate, and filtered. The filtrate was concentrated and purified by chromatography on silica gel (ethyl acetate/hexane, 1:1) to give 0.26 g of product as a yellow crystal (50% yield); mp: 223-224° C.; Anal. Calcd. for C₂₄H₂₄N₄O₄S: C, 62.05; H, 5.21; N, 12.06; S, 6.90. Found: C, 61.92; H, 5.04; N, 11.95; S, 7.03.

Step 6

A sealed reaction vessel containing the product from step 5 (0.2 g, 0.00043 mol) and 10 mL of liquid ammonia in 50 mL of absolute alcohol was heated at 90° C. and 250 PSI for 20 h. After cooling, the precipitate was filtered and air-dried to give 0.13 g of product as a yellow solid; mp: 237-238° C.; Anal. Calcd. for C₂₂H₂₁N₅O₃S: C, 60.67; H, 4.86; N, 16.08; S, 7.36. Found: C, 60.58; H, 4.93; N, 15.50; S, 7.10.

### Example 7

### 1-[4-(aminosulfonyl)phenyl]-6-[3-(methylamino)phenyl]-1H-indazole-3-carboxamide

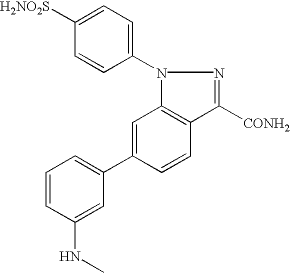

Step 1

To a cold suspension of the product from step 4 of Example 6 (1.08 g, 0.002 mol) and iodosobenzene in 20 mL of dry THF was added TMSN₃ slowly. The reaction mixture was stirred for 15 min. Then a mixture of ethyl acetate and sat. Na₂CO₃ was added and the aqueous phase was extracted with more ethyl acetate. The organic layer was washed with brine, dried over magnesium sulfate, and filtered. The filtrate was concentrated and purified by chromatography on silica gel (ethyl acetate/hexane, 1:3) to give 0.52 g of product as a yellow solid (50% yield); mp: 127-128° C.; Anal. Calcd. for C₂₉H₂₈N₄O₄S: C, 65.89; H, 5.34; N, 10.60; S, 6.07. Found: C, 65.65; H, 5.36; N, 10.48; S, 5.98.

Step 2

A solution of the product from step 1 (0.48 g, 0.0009 mol) in a mixture of TFA (15 mL) and water (5 mL) was heated at reflux for 2 h. The solvent was removed and the residue was basified with ammonium hydroxide solution and extracted with methylene chloride. The organic layer was washed with brine, dried over magnesium sulfate, and filtered. The filtrate was concentrated and purified by chromatography on silica gel (ethyl acetate/hexane, 6:4) to give 0.16 g of product as a yellow solid (39% yield); mp: 188-190° C.; Anal. Calcd. for C₂₃H₂₂N₄O₄S: C, 61.32; H, 4.92; N, 12.44; S, 7.12. Found: C, 61.53; H, 4.90; N, 11.70; S, 7.00.

Step 3

A sealed reaction vessel containing the product from step 2 (0.14 g, 0.0003 mol) and 10 mL of liquid ammonia in 50 mL of absolute alcohol was heated at 90° C. and 250 PSI for 20 h. After cooling, the precipitate was filtered and air-dried to give 0.12 g of product as a light yellow solid; mp: 159-160° C.; Anal. Calcd. for C₂₁H₁₉N₅O₃S: C, 59.84; H, 4.54; N, 16.62; S, 7.61. Found: C, 59.73; H, 4.55; N, 16.09; S, 7.46.

### Example 8

### 1-[4-(aminosulfonyl)phenyl]-1H-pyrazolo[4,3-c]pyridine-3-carboxamide

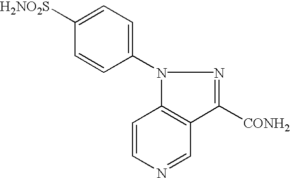

Step 1

To a solution of lithium bis(trimethylsilyl)amide (50 mL of 1.0 M in THF, 0.05 mol) in 100 mL of dry ether at −78° C. was added a solution of N-Bocpiperidone (10.0 g, 0.05 mol) in 25 mL of ether slowly. The reaction mixture was stirred at this temperature for 0.5 h. Then a solution of diethyl oxylate (7.5 g, 0.05 mol) in 25 mL of dry ether was added in one portion. The mixture was stirred overnight while warming up to room temperature. Water (400 mL) was added and the aqueous phase was neutralized with 1 N HCl, extracted with ethyl acetate. The organic layer was washed with brine, dried over magnesium sulfate, and filtered. The filtrate was concentrated to give 13.6 g of crude as a brown solid, which was used without further purification in the next step.

Step 2

A mixture of the crude product from Step 1 (3.6 g, 0.012 mol) and 4-sulfonamidophenylhydrazine hydrochloride (2.8 g, 0.012 mol) in 25 mL of acetic acid was heated at reflux for 6 h. After cooling, the solution was poured into 200 mL of water, basified with concentrated ammonia hydroxide. The aqueous phase was extracted with methylene chloride and the organic layer was washed with brine, dried over magnesium sulfate, and filtered. The filtrate was concentrated and the residue was triturated with ether to give 2.5 of product as a brown solid; mp: 144-146° C.; Anal. Calcd. for C₁₅H₁₈N₄O₄S: C, 51.42; H, 5.18; N, 15.99; S, 9.15. Found: C, 51.48; H,5.19; N, 16.09; S, 8.88.

Step 3

A mixture of the product from Step 2 (0.34 g, 0.001 mol) and 0.17 g of 10% Pd/C in 10 mL of nitrobenzene was stirred at reflux overnight. After cooling, the mixture was filtered through a pad of Celite® and the filtrate was concentrated. The crude was purified by chromatography on silica gel (ethyl acetate/hexane, 8:2) to give 0.19 g of product as a yellow solid; mp: 163-164° C.; Anal. Calcd. for C₁₅H₁₄N₄O₄S: C, 52.02; H, 4.07; N, 16.18; S, 9.26. Found: C, 52.20; H, 4.13; N, 15.62; S, 8.97.

Step 4

A sealed reaction vessel containing the product from Step 3 (0.23 g, 0.00064 mol) and 2.5 mL of liquid ammonia in 5 mL of absolute alcohol was heated at 110° C. for 20 h. After cooling, the precipitate was filtered and air-dried to give 0.16 g of product as a pale yellow crystal; mp: 301-302° C.; Anal. Calcd. for C₁₃H₁₁N₅O₃S: C, 49.21; H, 3.49; N, 22.07; S, 10.10. Found: C, 48.85; H, 3.47; N, 21.86; S, 10.12.

### Example 9

### 1-[4-(aminosufony)phenyl]-6-methyl-1H-indazole-3-carboxamide

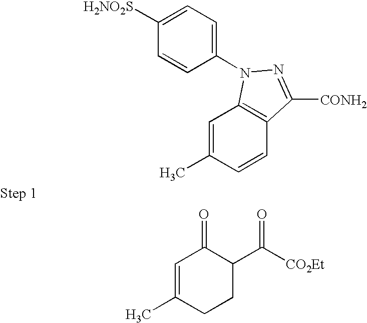

To a stirred solution of sodium ethoxide in ethanol at room temperature, prepared from 2.1 g (0.090mol) of sodium metal and 30 ml of ethanol, was added a solution of commercially available 3-methylcyclohex-2-en-1-one (10 g, 0.09 mol) and diethyl oxalate (13.2 g, 0.09 mol) in ethanol (30 ml). When the addition was completed (30 min.), the reaction was stirred at room temperature overnight. The reaction was acidified with 3N hydrochloric acid (150 ml) and extracted with ethyl acetate. The organic layer was dried over magnesium sulfate and the crude product was chromatographed on silica gel using mixtures of ethyl acetate and hexane as the eluents. The purified diketo ester was isolated as an oil. Anal. Calcd. for C₁₁H₁₄O₄: C, 62.85; H, 6.71. Found: C, 62.39; H, 6.80.

Step 2

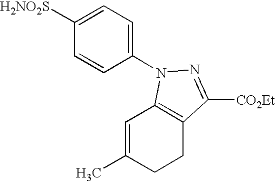

A solution of diketo ester from the preceding step (5.0 g, 0.024 mol) and 4-sulfonamidophenylhydrazine hydrochloride (5.1 g, 0.024 mol) in ethanol (100 ml) was refluxed for 6 hr. The reaction solution was cooled and the precipitate filtered to give the product dihydroindazole (7.3 g, 84%) suitable for use without further purification. Anal. Calcd. for C₁₇H₁₉N₃O₄S: C, 56.50; H, 5.30; N, 11.63. Found: C, 56.40; H, 5.37; N, 11.56.

Step 3

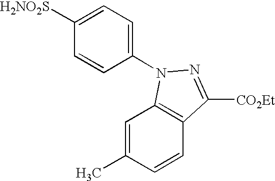

The dihyroindazole from the preceding example (7.25 g, 00020 mol) and 10% Pd/C (3.4 g) were combined in cumene (110 ml) and refluxed with stirring under a nitrogen atmosphere for 48 hrs. The reaction mixture was cooled to 75° and the catalyst filtered using Celite, taking care to wash the filter cake thoroughly with ethyl acetate and then methanol. The filtrate was evaporated and the residue triturated with 1:1 ethyl acetate:hexane and filtered. The product (3.1 g, 43%) was used without further purification. Anal. Calcd. for C₁₇H₁₇N₃O₄S: C, 56.81; H, 4.77; N, 11.69. Found: C, 56.51; H, 4.69; N, 11.40.

Step 4

A solution of the ester from the preceding example (2.6 g, 0.0073 mol) in ethanol (50 ml) and ammonia (25 ml, liquid) was heated in a Parr shaker at 90° and 300 psi for 18 hr. The reaction was cooled, the pressure released and the solvent evaporated to yield a crude solid. This crude product was recrystallized from ethanol and water to give the purified indazole (1.85 g, 77%), m. p. 251-252°. Anal. Calcd. for C₁₅H₁₄N₄O₃S: C, 54.53; H, 4.27; N, 16.96. Found: C, 54.15; H, 4.33; N, 16.60.

### Example 10

### 1-[4-(aminosulfonyl)phenyl]-6-phenyl-1H-indazole-3-carboxamide

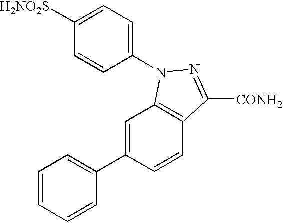

3-Phenylcyclohex-2-en-1-one may be prepared according the to the procedure described by G. F. Woods and I. W. Tucker (J. Am. Chem. Soc., 70, 2174 (1948). Starting with this ketone, the target indazole was synthesized using the procedures described in Example 9 for 1-[4-(aminosulfonyl)phenyl]-6-methyl-1H-indazole-3-carboxamide. The product of this example had m. p. 232-234+. Anal. Calcd. for C₂₀H₁₆N₄O₃S: C, 61.21; H, 4.11; N, 14.28. Found: C, 61.18; H, 4.01; N, 14.11.

### Example 11

### 1-[4-(aminosulfonyl)phenyl]-6-(3-methoxyphenyl)-1H-indazole-3-carboxamide

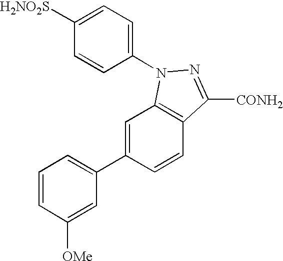

The starting ketone, 3-(3-methoxyphenyl)cyclohex-2-en-1-one, may be prepared according the to the procedure described by G. F. Woods and I. W. Tucker (J. Am. Chem. Soc., 70, 2174 (1948) for 3-phenylcyclohex-2-en-1-one. Starting with the methoxyphenyl ketone, the target indazole was synthesized using the procedures described in Example 9 for 1-(4-sulfonamidophenyl)-3-carboxyamido-7-methylindazole. The product of this example had m. p. 229-230°. Anal. Calcd. for C₂₁H₁₈N₄O₄S: C, 59.70; H, 4.29; N, 13.26. Found: C, 59.82; H, 4.67; N, 12.97.

### Example 12

### 1-[4-(aminosulfonyl)phenyl]-6-benzyl-1H-indazole-3-carboxamide

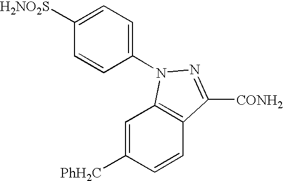

The starting ketone, 3-benzylcyclohex-2-en-1-one, may be prepared according the to the procedure described by G. F. Woods and I. W. Tucker (J. Am. Chem. Soc., 70, 2174 (1948) for 3-phenylcyclohex-2-en-1-one. Starting with the benzyl ketone, the target indazole was synthesized using the procedures described in Example 9 for 1-[4-(aminosulfonyl)phenyl]-6-methyl-1H-indazole-3-carboxamide. The product of the current example had m. p. 207-209°. Anal. Calcd. for C₂₁H₁₈N₄O₃S+0.5 H₂O: C, 60.71; H, 4.61; N, 13.49. Found: C, 60.96; H, 4.50; N, 13.12.

### Example 13

### 1-[4-(aminosulfonyl)phenyl]-6-ethoxy-1H-indazole-3-carboxamide

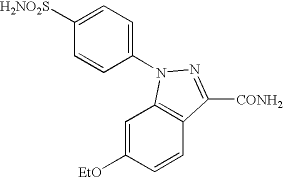

The starting ketone, 3-ethoxycyclohex-2-en-1-one, was purchased from a commercial source. Starting with this ketone, the target indazole was synthesized using the procedures described in Example 9 for 1-(4-sulfonamidophenyl)-3-carboxyamido-7-methylindazole. The product of the current example had m. p. 213-214°. Anal. Calcd. for C₁₆H₁₆N₄O₄S+H₂O: C, 50.79; H, 4.79; N, 14.81. Found: C, 50.49; H, 5.03; N, 14.60.

### Example 14

### 1-[4-(aminosulfonyl)phenyl]-6-ethyl-1H-indazole-3-carboxamide

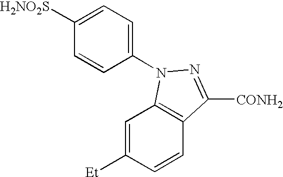

The starting ketone, 3-ethylcyclohex-2-en-1-one, may be prepared according the to the procedure described by G. F. Woods and I. W. Tucker (J. Am. Chem. Soc., 70, 2174 (1948) for 3-phenylcyclohex-2-en-1-one except that ethylmagnesium bromide was used in place of phenylmagnesium bromide. Starting with the ethyl ketone, the target indazole was synthesized using the procedures described in Example 9. The product of the current example had m. p. 249-251°. Anal. Calcd. for C₁₆H₁₆N₄O₃S+0.5 H₂O: C, 54.38; H, 4.85; N, 15.85. Found: C, 54.43; H, 5.09; N, 15.69.

### Example 15

### 1-[4-(aminosulfonyl)phenyl]-6-pyridin-3-yl-1H-indazole-3-carboxamide

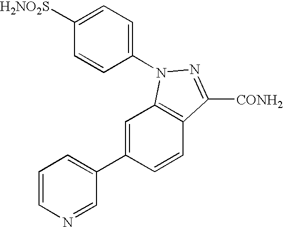

The required starting ketone 3-(3-pyridyl)cyclohex-2-en-1-one was prepared according to the procedure described in U.S. Pat. No. 4,026,900. Starting with this ketone, the target indazole was synthesized using the procedures described in Example 9 for 1-(4-sulfonamidophenyl)-3-carboxyamido-7-methylindazole except that the base was potassium tert.-butoxide and the solvent was tetrahydrofuran. The product of the current example had m. p. 310-313°. Anal. Calcd. for C₁₉H₁₅N₅O₃S+0.5 H₂O.: C, 56.71; H, 4.01; N, 17.40. Found: C, 56.24; H, 4.51; N, 16.91.

### Example 16

### 1-[4-(aminosulfonyl)phenyl]-6-(2-hydroxyphenyl)-1H-indazole-3-carboxamide

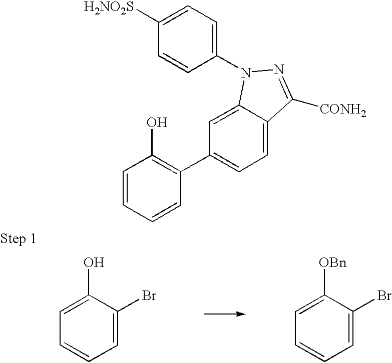

A mixture of 20.0 g (116 mmoles) of 2-bromophenol, 4.0 g (100 mmoles) of sodium hydroxide, 1 g of tetraethylammonium chloride hydrate, 14 ml (20g, 116 mmoles) of benzyl bromide, 100 ml of dichloromethane, and 100 ml of water was stirred rapidly at reflux for a total of 8 h. After cooling, the layers were separated, the aqueous layer was extracted with dichloromethane, and the combined organic extracts dried over sodium sulfate. The solution was filtered and concentrated, and the residue purified by simple distillation to give 23.5 g of the title compound as a water-white solid. The structure was supported by ¹H NMR and by ¹³C NMR.

Step 2

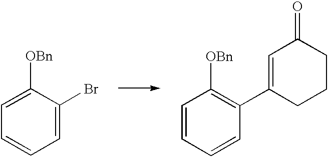

To a solution of 23.5 g (89.4mmoles) of the title product of step 1 in 100 ml of dry tetrahydrofuran was added 2.17 g (89.4 mmoles) of magnesium turnings and a few crystals of iodine. After reaction was initiated, reflux was maintained with external heating for 2 h. The mixture was cooled in an ice bath, and a solution of 12.5 g (89.4 mmoles) of 3-ethoxycyclohex-2-en-1-one in 25 ml of tetrahydrofuran was added. After stirring overnight at room temperature, brine was added and the mixture extracted with ethyl acetate. The combined organic extracts were washed with brine, dried over sodium sulfate, filtered, and concentrated. Chromatography of the residue over silica gel using 35% ethyl acetate—hexane as eluent gave the title compound, 11.0 g, as a very light yellowish oil. The structure was supported by ¹H NMR.

Step 3

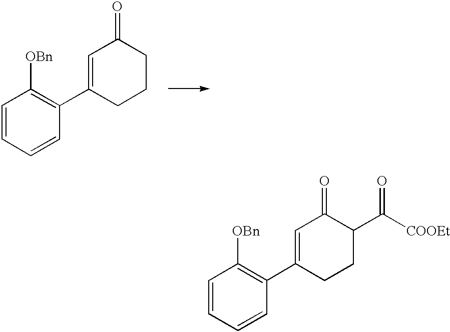

To a mixture of 38 ml of 1M sodium bis(trimethylsilyl)amide and 50 ml of tetrahydrofuran stirring in a Dry Ice—2-propanol bath under argon was added dropwise a solution of 10.6 g (38.1 mmoles) of the title product of step 2 in 100 ml of tetrahydrofuran. After the addition the mixture was stirred for 25 min, and then 5.2 ml (5.6 g) of diethyl oxalate was added, and stirring continued overnight while warming to room temperature. The mixture was diluted with ethyl acetate, and then washed with ice-cold 3N aqueous hydrochloric acid. The organic layer was washed with brine, dried over sodium sulfate, filtered, and evaporated to give the title compound, 14.8 g, as a reddish oil. The structure was supported by ¹H NMR.

Step 4

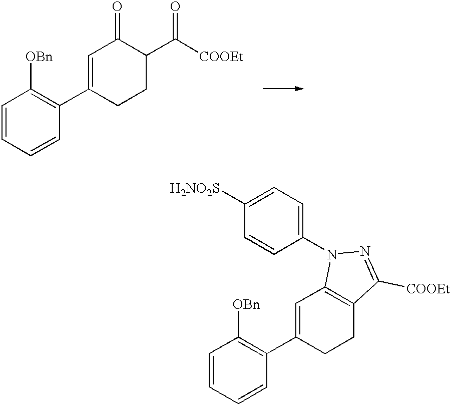

A mixture of 2.03 g (5.36 mmoles) of the title product of Step 3 and 1.20 g (5.36 mmoles) of 4-sulfonamidophenylhydrazine in 50 ml of acetic acid was stirred at reflux for 2 h and then cooled. After cooling, the mixture was concentrated and the residue chromatographed over silica gel using 50% ethyl acetate—hexane as eluent to give the title compound, 2.11 g, as a yellow foam. The structure was supported by ¹H NMR.

Step 5

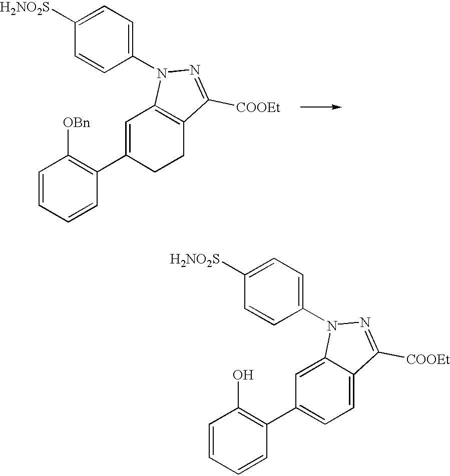

A solution of 2.11 g (4.00 mmoles) of the title product of step 4 in 50 ml of cumene and 20 ml of N-methylpyrrolidone was treated with about 100 mg of 5% palladium on carbon, and then stirred at reflux for 2 h. After cooling, the mixture was filtered and concentrated. Chromatography of the residue over silica gel using 50% ethyl acetate—hexane as eluent gave the title compound, 230 mg, as a pale yellow solid. The structure was supported by ¹H NMR.

Step 6

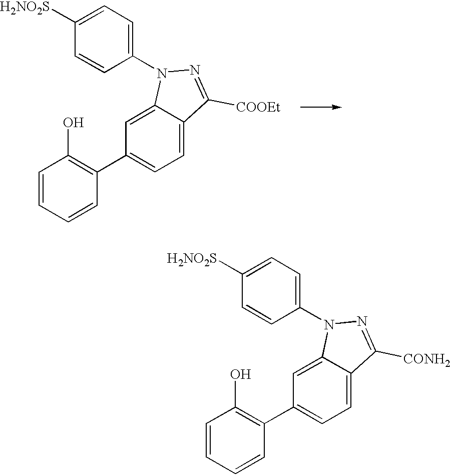

A mixture of 230 mg (0.530 mmole) of the title product of step 5 in ethanol and liquid ammonia in a pressure apparatus was heated to around 100° C. for 20 h. After cooling, the mixture was evaporated, and the residue triturated with ethyl acetate containing some methanol to give, after filtration and drying, the title compound (100 mg) as a light grayish solid. Anal. Calc'd. for C₂₀H₁₆N₄O₄S+1.5H₂O (MW 435.46): C, 55.16, H, 3.70, N, 12.87. Found: C, 55.04, H, 4.13, N, 13.22. DSC 251° C., 287° C.

### Example 17

### 1-[4-(aminosulfonyl)phenyl]-6-(3-hydroxyphenyl)-1H-indazole-3-carboxamide

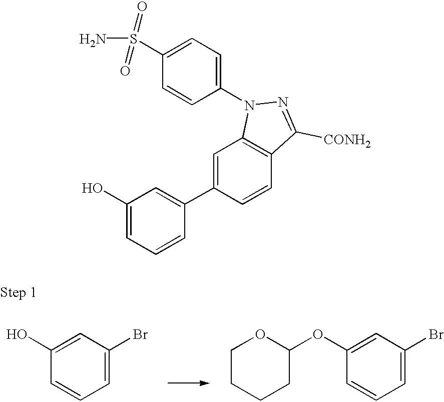

To a solution of 25.0 g (145 mmoles) of 3-bromophenol in 400 ml of dry tetrahydrofuran was added 20 ml (18 g, 220 mmoles) of dihydropyran and then 300 mg of p-toluenesulfonic acid monohydrate. The resulting solution was stirred at room temperature for three days, after which 75 ml of 1M aqueous sodium hydroxide was added, and the volatiles evaporated. The residue was partitioned between water and diethyl ether, and the organic layer dried over sodium sulfate. After filtration and concentration, the residue was distilled under high vacuum to give the title compound, 26.4 g, as a water white liquid. The structure was supported by ¹H NMR.

Step 2

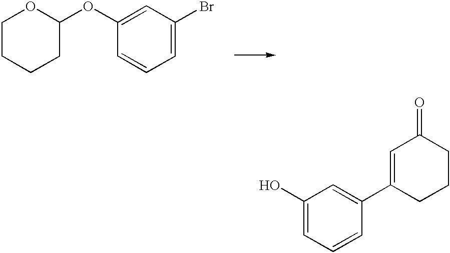

To 2.73 g (112 mmoles) of magnesium turnings in 50 ml of dry tetrahydrofuran was added a few ml of a solution of 26.3 g (102 mmoles) of the title product of step 1 in 25 ml of tetrahydrofuran. A few crystals of iodine and then 0.4 ml of a solution of 2M benzylmagnesium chloride in tetrahydrofuran were added, and the mixture warmed to gentle reflux. About half of the aryl bromide solution was then added, and the heat source removed, with reflux continuing spontaneously. The remaining aryl bromide solution was then added, and after reflux subsided, was continued with external heating for 1 hour. The mixture was cooled to room temperature, and a solution of 14.3 g (102 mmoles) of 3-ethoxycyclohex-2-ene-1-one in 25 ml of tetrahydrofuran was added. After stirring overnight, the supernatant was decanted from unreacted magnesium, 100 ml of 3N aqueous hydrochloric acid were added, and the mixture stirred for 0.5 h. Brine was added, the mixture extracted with diethyl ether, and the combined organic extracts dried over sodium sulfate. The solution was filtered and concentrated. Trituration of the residue with dichloromethane gave the title compound, 5.77 g, as a pale yellowish crystalline solid.

Anal. Calc'd. for C₁₂H₁₂O₂(MW 188.23): C, 76.57, H, 6.43. Found: C, 76.27, H, 6.67.

Step 3

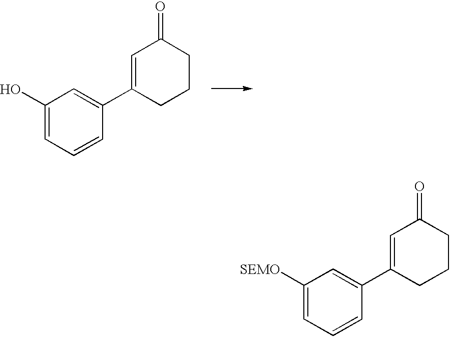

To a suspension of 5.77 g (30.7 mmoles) of the title product of step 2 in 100 ml of dry tetrahydrofuran stirring in a Dry Ice—2-propanol bath under argon was added dropwise 32 ml of a 1.0M solution of sodium bis(trimethylsilyl)amide in tetrahydrofuran. After 30 min, a solution of 5.37 g (32.3 mmoles) of 2-(trimethylsilyl)ethoxymethyl chloride in 15 ml of tetrahydrofuran was added, and the mixture stirred while allowing to warm to room temperature over 1h. A further 1.5 ml of 2-(trimethylsilyl)ethoxymethyl chloride was added, and stirring continued for 30 min. Water was added, and the mixture extracted twice with ethyl acetate. The combined organic extracts were dried over sodium sulfate, filtered, and concentrated to give the title compound, 7.12 g, as a water-white oil. The structure was supported by ¹H NMR.

Step 4

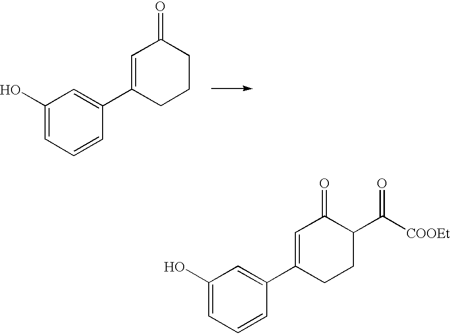

To 22.4 ml of a 1.0M solution of sodium bis(trimethylsilyl)amide in tetrahydrofuran and 90 ml of tetrahydrofuran stirring in a Dry Ice—2-propanol bath under argon was added dropwise a solution of 7.12 g (22.4 mmoles) of the title product of step 3 in 40 ml of tetrahydrofuran. After stirring for 15 min, a solution of 3.26 g (22.4 mmoles) of diethyl oxalate was added, and the mixture stirred whole allowing to warm to room temperature over 2 h. The mixture was diluted with ethyl acetate, and washed with 1.5M aqueous hydrochloric acid. The aqueous layer was extracted with ethyl acetate, the combined organic extracts washed with brine, then dried over sodium sulfate, filtered, and concentrated to give the title compound, 9.14 g, as an orange oil. The structure was supported by ¹H NMR.

Step 5

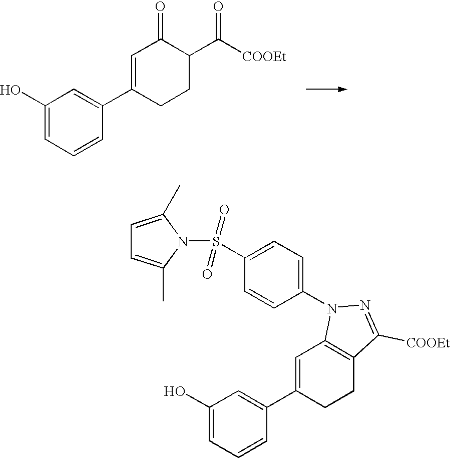

A mixture of 9.14 g (21.8 mmoles) of the title product of step 4 and 5.79 g (21.8 mmoles) of 4-[(2,5-dimethylpyrrolyl)sulfonyl]phenylhydrazine in 180 ml of acetic acid was stirred at reflux for 2 h, and then cooled. Acetic acid was removed by azeotropic distillation with toluene. Chromatography of the residue over silica gel using 40% ethyl acetate—hexane as eluent gave the title compound, 8.49 g, as a yellow-orange solid. The structure was supported by ¹H NMR.

Step 6

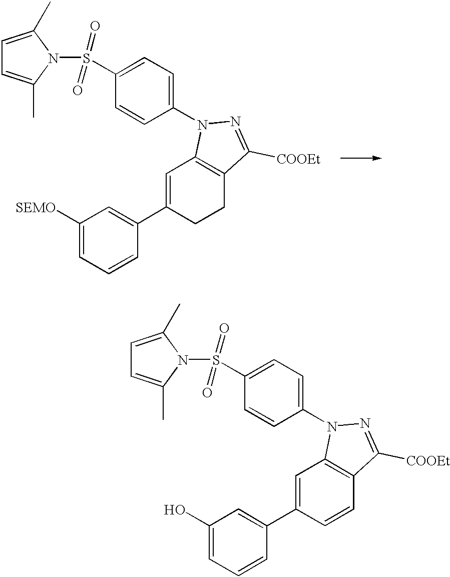

To a solution of 3.61 g (5.57 mmoles) of the title product of Step 5 in 100 ml of cumene and 10 ml of N-methylpyrrolidinone was added a large spatula end of 10% palladium on carbon. The mixture was stirred at reflux for 2 h then cooled and filtered through diatomaceous earth. After concentration, the residue was chromatographed over silica gel using 50% ethyl acetate—hexane as eluent followed by trituration with dichloromethane to give the title compound, 0.89 g, as an off-white solid. The structure was supported by 1H NMR.

Step 7

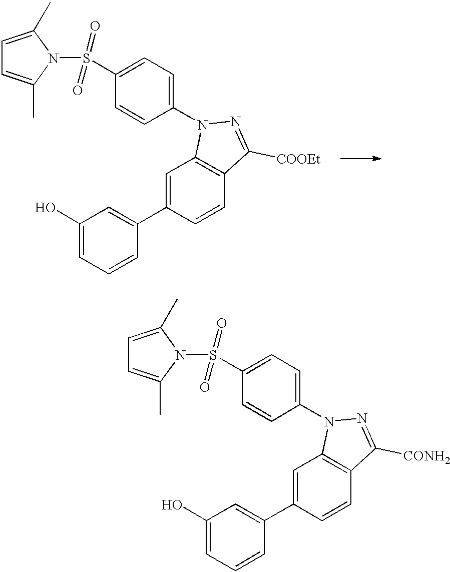

To a suspension of 300 mg (0.581 mmole) of the title product of step 6 in 5 ml of methanol was added 15 ml of concentrated ammonium hydroxide, and 6 ml of dimethylformamide. The resulting mixture was kept at room temperature for 6 days. The methanol was evaporated by rotary evaporation and drying completed by lyophilization to give the title compound, which was used without further manipulation.

Step 8

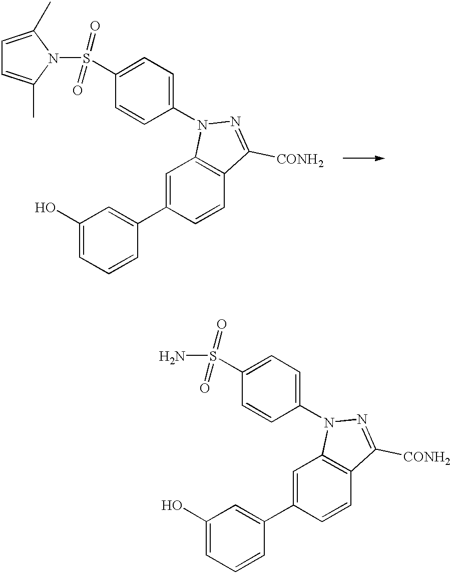

Trifluoroacetic acid, 15 ml, and then water, 5 ml, were added to the title product of step 7. The resulting mixture was brought to reflux with stirring and so maintained for 0.5 h. After cooling, the mixture was added to saturated aqueous sodium bicarbonate containing solid sodium bicarbonate. The supernatant was decanted, and the solids washed with 5:1 dichloromethane—methanol. The organic extracts and decanted supernatant were shaken in a separatory funnel, with a flocculent solid appearing. The solid was isolated by filtration and washed with water. The residue was boiled with methanol then cooled. Filtration gave the title compound, 62 mg, as a tan solid.

Anal. Calc'd. for C₂₀H₁₆N₄O₄S+H₂O (MW 426.45): C, 56.33, H, 3.78, N, 13.14. Found: C, 56.22, H, 4.02, N, 12.49. DSC 288° C.

### Example 18

### 6-(2-hydroxyphenyl)-1-[4-(methylsulfonyl)phenyl]-1H-indazole-3-carboxamide

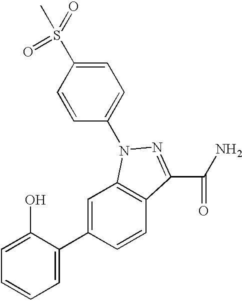

### Example 19

### 1-[3(aminosulfonyl)phenyl]-6-phenyl-1H-indazole-3-carboxamide

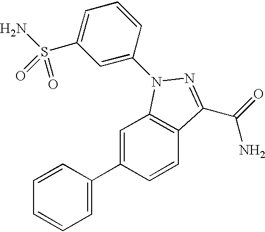

Table 1 shows the bioactivity for the exemplified compounds as measured in the IKK heterodimer Resin Enzyme Assay expressed as IC50.

TABLE 1

| COMPOUND | STRUCTURE | EXAMPLE | HetD |
|---|---|---|---|
| 1-[4-(aminosulfonyl)phenyl]-6- (4-methoxyphenyl)-1H- indazole-3-carboxamide | 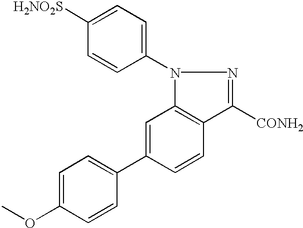 | Example 1 | >100 μM |
| 1-[4-(aminosulfonyl)phenyl]-6- (4-fluorophenyl)-1H-indazole-3- carboxamide | 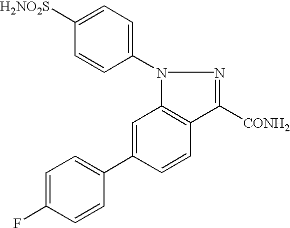 | Example 2 | 10 = 100 μM |
| 1-[4-(aminosulfonyl)phenyl]-6- (3-methylphenyl)-1H-indazole- 3-carboxamide | 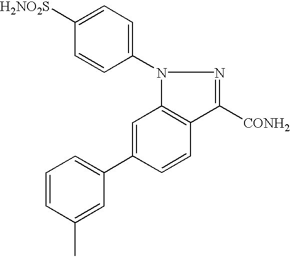 | Example 3 | 1 = 10 μM |
| 1-[4-(aminosulfonyl)phenyl]-6- (4-tert-butylphenyl)-1H- indazole-3-carboxamide | 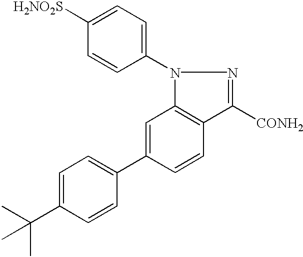 | Example 4 | >100 μM |
| 1-[4-(aminosulfonyl)phenyl]-6- (4-fluoro-3-methylphenyl)-1H- indazole-3-carboxamide | 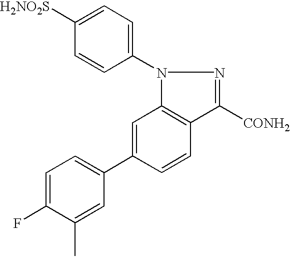 | Example 5 | 10 = 100 μM |
| 1-[4-(aminosulfonyl)phenyl]-6- [3-(dimethylamino)phenyl]-1H- indazole-3-carboxamide | 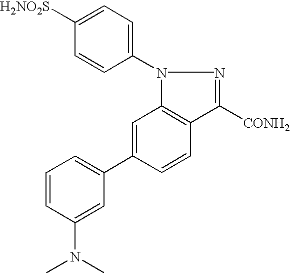 | Example 6 | >100 μM |
| 1-[4-(aminosulfonyl)phenyl]-6- [3-(methylamino)phenyl]-1H- indazole-3-carboxamide | 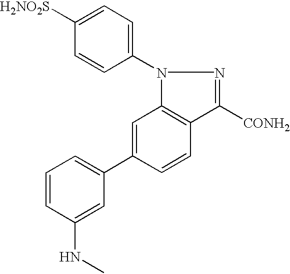 | Example 7 | 1 = 10 μM |
| 1-[4-(aminosulfonyl)phenyl]- 1H-pyrazolo[4,3-c]pyridine-3- carboxamide | 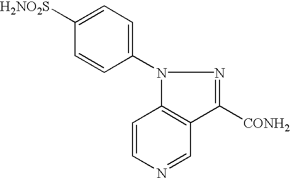 | Example 8 | 10 = 100 μM |
| 1-[4-(aminosulfonyl)phenyl]-6- methyl-1H-indazole-3- carboxamide | 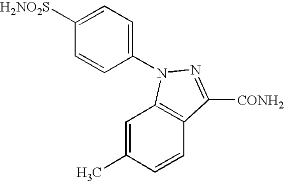 | Example 9 | 1 = 10 μM |
| 1-[4-(aminosulfonyl)phenyl]-6- phenyl-1H-indazole-3- carboxamide | 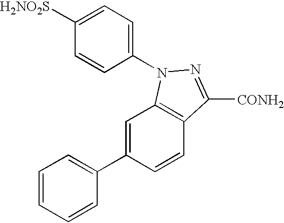 | Example 10 | 1 = 10 μM |
| 1-[4-(aminosulfonyl)phenyl]-6- (3-methoxyphenyl)-1H- indazole-3-carboxamide | 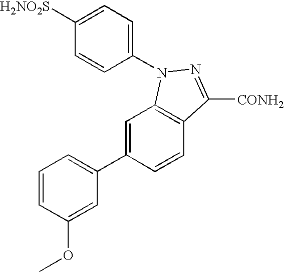 | Example 11 | 1 = μM |
| 1-[4-(aminosulfonyl)phenyl]-6- benzyl-1H-indazole-3- carboxamide | 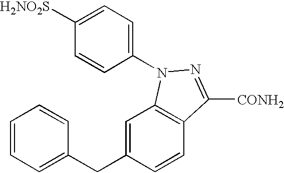 | Example 12 | 1 = 10 μM |
| 1-[4-(aminosulfonyl)phenyl]-6- ethoxy-1H-indazole-3- carboxamide | 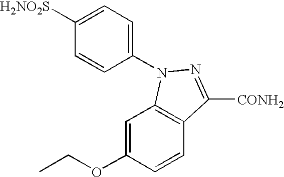 | Example 13 | 1 = 10 μM |
| 1-[4-(aminosulfonyl)phenyl]-6- ethyl-1H-indazole-3- carboxamide | 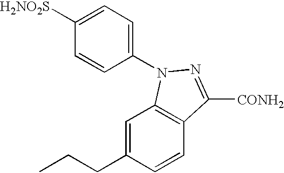 | Example 14 | 10 = 100 μM |
| 1-[4-(aminosulfonyl)phenyl]-6- pyridin-3-yl-1H-indazole-3- carboxamide | 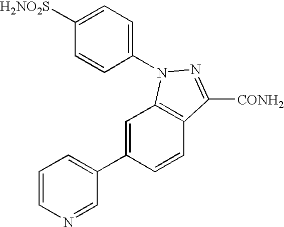 | Example 15 | 1 = 10 μM |
| 1-[4-(aminosulfonyl)phenyl]-6- (2-hydroxyphenyl)-1H- indazole-3-carboxamide | 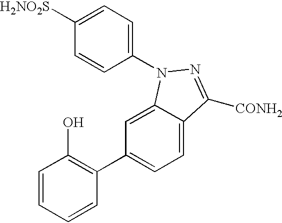 | Example 16 | 1 = 10 μM |
| 1-[4-(aminosulfonyl)phenyl]-6- (3-hydroxyphenyl)-1H- indazole-3-carboxamide | 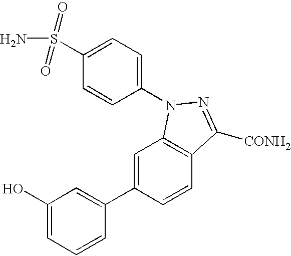 | Example 17 | 1 = 10 μM |
| 6-(2-hydroxyphenyl)-1-[4- (methylsulfonyl)phenyl]-1H- indazole-3-carboxamide | 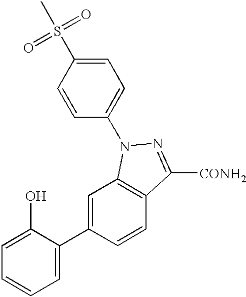 | Example 18 | 1 = 10 μM |
| 1-[3-(aminosulfonyl)phenyl]-6- phenyl-1H-indazole-3- carboxamide | 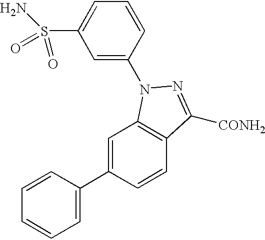 | Example 19 | 10 = 100 μM |

### Biological Evaluation

Materials

SAM² ™ 96 Biotin capture plates were from Promega. Anti-FLAG affinity resin, FLAG-peptide, NP-40 (Nonidet P-40), BSA, ATP, ADP, AMP, LPS (E. coli serotype 0111:B4), and dithiothreitol were obtained from Sigma Chemicals. Antibodies specific for NEMO (IKKγ) (FL-419), IKK1 (H-744), IKK2 (H-470) and IκBα(C-21) were purchased from Santa Cruz Biotechnology. Ni-NTA resin was purchased from Qiagen. Peptides were purchased from American Peptide Company. Protease inhibitor cocktail tablets were from Boehringer Mannheim. Sephacryl S-300 column was from Pharmacia LKB Biotechnology. Centriprep-10 concentrators with a molecular weight cutoff of 10 kDa and membranes with molecular weight cut-off of 30 kDa were obtained from Amicon. [γ-³³P] ATP (2500 Ci/mmol) and [γ-³³P] ATP (6000 Ci/mmol) were purchased from Amersham. The other reagents used were of the highest grade commercially available.

Cloning and Expression

cDNAs of human IKK1 and IKK2 were amplified by reverse transcriptase-polymerase chain reaction from human placental RNA (Clonetech). hIKK1 was subcloned into pFastBac HTa (Life Technologies) and expressed as N-terminal His₆-tagged fusion protein. The hIKK2 cDNA was amplified using a reverse oligonucleotide primer which incorporated the peptide sequence for a FLAG-epitope tag at the C-terminus of the IKK2 coding region (DYKDDDDKD). The hIKK2:FLAG cDNA was subcloned into the baculovirus vector pFastBac. The rhIKK2 (S177S, E177E) mutant was constructed in the same vector used for wild type rhIKK2 using a QuikChange™ mutagenesis kit (Stratagene) Viral stocks of each construct were used to infect insect cells grown in 40L suspension culture. The cells were lysed at a time that maximal expression and rhIKK activity were demonstrated. Cell lysates were stored at −80° C. until purification of the recombinant proteins was undertaken as described in the succeeding sections.

Enzyme Isolation

All purification procedures were carried out at 4° C. unless otherwise noted. Buffers used are: buffer A: 20 mM Tris-HCl, pH 7.6, containing 50 mM NaCl, 20 mM NaF, 20 mM β-Glycerophosphate, 500 uM sodiumortho-vanadate, 2.5 mM metabisulfite, 5 mM benzamidine, 1 mM EDTA, 0.5 mM EGTA, 10% glycerol, 1 mM DTT, 1×Complete™ protease inhibitors; buffer B: same as buffer A, except 150 mM NaCl, and buffer C: same as buffer A, except 500 mM NaCl.

Isolation of rhIKK1 Homodimer

Cells from an 8 liter fermentation of baculovirus-expressed IKK1 tagged with His peptide were centrifuged and the cell pellet (MOI 0.1, I=72 hr) was re-suspended in 100 ml of buffer C. The cells were microfluidized and centrifuged at 100,000×g for 45 min. The supernatant was collected, imidazole added to the final concentration of 10 mM and incubated with 25 ml of Ni-NTA resin for 2 hrs. The suspension was poured into a 25 ml column and washed with 250 ml of buffer C and then with 125 ml of 50 mM imidazole in buffer C. rhIKK1 homodimer was eluted using 300 mM imidazole in buffer C. BSA and NP-40 were added to the enzyme fractions to the final concentration of 0.1%. The enzyme was dialyzed against buffer B, aliquoted and stored at −80° C.

Isolation of rhIKK2 Homodimer

A 10 liter culture of baculovirus-expressing IKK2 tagged with FLAG peptide was centrifuged and the cell pellet (MOI=0.1 and I=72 hrs) was re-suspended in buffer A. These cells were microfluidized, and centrifuged at 100,000×g for 45 min. Supernatant was passed over a G-25 column equilibrated with Buffer A. Protein peak was collected and incubated with anti-FLAG affinity resin on a rotator overnight in buffer B. The resin was washed in batch with 10-15 bed volumes of buffer C. Washed resin was poured into a column and rhIKK2 homodimer was eluted using 5 bed volumes of buffer B containing FLAG peptide. 5 mM DTT, 0.1% NP-40 and BSA (concentrated to 0.1% in final amount) was added to the eluted enzyme before concentrating in using an Amicon membrane with a molecular weight cut-off of 30 kDa. Enzyme was aliquoted and stored at −80° C.

Isolation of rhIKK1/IKK2 Heterodimer

The heterodimer enzyme was produced by coinfection in a baculovirus system (FLAG IKK2/IKK1 His; MOI=0.1 and I=72 hrs). Infected cells were centrifuged and the cell pellet (10.0 g) was suspended in 50 ml of buffer A. The protein suspension was microfluidized and centrifuged at 100,000×g for 45 min. Imidazole was added to the supernatant to a final concentration of 10 mM. The protein was allowed to bind 25 ml of Ni-NTA resin by mixing for 2 hrs. The protein-resin slurry was poured into a 25 ml column and washed with 250 ml of buffer A containing 10 mM imidazole followed by 125 ml of buffer A containing 50 mM imidazole. Buffer A, containing 300 mM imidazole, was then used to elute the protein. A 75 ml pool was collected and NP-40 was added to a final concentration of 0.1%. The protein solution was then dialyzed against buffer B. The dialyzed heterodimer enzyme was then allowed to bind to 25 ml of anti-FLAG M2 agarose affinity gel overnight with constant mixing. The protein-resin slurry was then centrifuged for 5 min at 2,000 rpm. The supernatant was collected and the resin re-suspended in 100 ml of buffer C containing 0.1% NP-40. The resin was washed with 375 ml of buffer C containing 0.1% NP-40. The protein-resin was poured into a 25 ml column and the enzyme eluted using buffer B containing FLAG peptide. Enzyme fractions (100 ml) were collected and concentrated to 20 ml using an Amicon membrane with molecular weight cut-off of 30 kDa. Bovine serum albumin was added to the concentrated enzyme to final concentration of 0.1%. The enzyme was then aliquoted and stored at −80° C.

Cell Culture

The wild type (wt) human pre-B cell line, 70Z/3, and its mutant, 1.3E2, were generously provided by Dr. Carol Sibley. Wt 70Z/3 and 1.3E2 cells were grown in RPMI 1640 (Gibco) supplemented with 7% defined bovine serum (Hyclone) and 50 μM 2-mercaptoethanol. Human monocytic leukemia THP-1 cells, obtained from ATCC, were cultured in RPMI 1640 supplemented with 10% defined bovine serum, 10 mM HEPES, 1.0 mM sodium pyruvate and 50 μM 2-mercaptoethanol. For experiments, cells were plated in 6 well plates at 1×10⁶ cells/ml in fresh media. Pre-B cells were stimulated by the addition of 10 μg/ml LPS for varying lengths of time ranging from 0-4 hr. THP-1 cells were stimulated by the addition of 1 μg/ml LPS for 45 minutes. Cells were pelleted, washed with cold 50 mM sodium phosphate buffer, pH 7.4 containing 0.15 M NaCl and lysed at 4° C. in 20 mM Hepes buffer, pH 7.6 containing 50 mM NaCl, 1 mM EDTA, 1 mM EGTA, 1 mM sodium orthovanadate, 10 mM β-glycerophosphate, 1 mM NaF, 1 mM PMSF, 1 mM DTT and 0.5% NP40 (lysis buffer). The cytosolic fractions obtained following centrifugation at 10,000×g were stored at −80° C. until used.

Immunoprecipitation and Western Blotting

SF9 cells paste containing rhIKKs were centrifuged (100,000×g, 10 min) to remove debris. rhIKKs were immunoprecipitated (100 μg of cell paste) from the cell supernatant using 3 μg of anti-NEMO antibody (FL-419), followed by coupling to protein A sepharose beads. rhIKKs were also immunoprecipitated from affinity chromatography purified protein preparations (1 μg) using anti-FLAG, anti-His or anti-NEMO antibodies (1-4 μg) followed by protein A sepharose coupling. The native, human IKK complex was immunoprecipitated from THP-1 cell homogenates (300 μg/condition) using the anti-NEMO antibody. Immune complexes were pelleted and washed 3 times with 1 ml cold lysis buffer. Immunoprecipitated rhIKKs were chromatographed by SDS-PAGE (8% Tris-glycine) and transferred to nitrocellulose membranes (Novex) and detected by chemiluminescense (SuperSignal) using specific anti-IKK antibodies (IKK2 H-470, IKK1 H-744). Native IKK2, IκBα and NEMO proteins from cytosolic lysates (20-80 μg) were separated by SDS-PAGE and visualized by chemiluminescense using specific antibodies.

Phosphatase Treatment

Immunoprecipitated rhIKKs were washed 2 times in 50 mM Tris-HCl, pH 8.2 containing 0.1 mM EDTA, 1 mM DTT, 1 mM PMSF and 2 mM MnCl₂ and resuspended in 50 μl. Phosphatase (λPPase, 1000 U) was pre-diluted in the same buffer and added to the IKK samples. Following incubation at room temperature for 30 minutes with intermittent mixing, cold lysis buffer was added to the tubes to stop the reaction. After several washes, 10% of the beads were removed for Western analysis, and the remaining material was pelleted and resuspended in 100 μl of the buffer used for the in vitro kinase assay.

IKKαSAM Enzyme Assay

IKKα kinase activity was measured using a biotinylated IκBα peptide (Gly-Leu-Lys-Lys-Glu-Arg-Leu-Leu-Asp-Asp-Arg-His-Asp-Ser₃₂-Gly-Leu-Asp-Ser₃₆-Met-Lys-Asp-Glu-Glu), a SAM² ™ 96 Biotin capture plate, and a vacuum system. The standard reaction mixture contained 5 μM biotinylated IκBα peptide, 1 μM [γ-³³P] ATP (about 1×10⁵ cpm), 1 mM DTT, 50 mM KCl, 2 mM MgCl₂, 2 mM MnCl₂, 10 mM NaF, 25 mM Hepes buffer, pH. 7.6 and enzyme solution (1-10 μl) in a final volume of 50 μl. After incubation at 25° C. for 30 min, 25 μl of the reaction mixture was withdrawn and added to a SAM²™ 96 Biotin capture 96-well plate. Each well was then washed successively with 800 μl 2 M NaCl, 1.2 ml of NaCl containing 1% H₃PO₄, 400 μl H₂O, and 200 μl 95% ethanol. The plate was allowed to dry in a hood at 25° C. for 1 hr and then 25 μl of scintillation fluid (Microscint 20) was added to each well. Incorporation of [γ-³³P] ATP was measured using a Top-Count NXT (Packard). Under each assay condition, the degree of phosphorylation of IκBα peptide substrate was linear with time and concentration for all purified enzymes. Results from the biotinylated peptide assay were confirmed by SDS-PAGE analysis of kinase reaction utilizing a GST-IκBα₁₋₅₄ and [γ-³³P] ATP. The resulting radiolabeled substrate was quantitated by Phosphoimager (Molecular Dynamics). An ion exchange resin assay was also employed using [γ-³³P] ATP and GST-IκBα₁₋₅₄ fusion protein as the substrates. Each assay system yielded consistent results in regard to Kₘ and specific activities for each of the purified kinase isoforms. One unit of enzyme activity was defined as the amount required to catalyze the transfer of 1 nmole of phosphate from ATP to IκBα peptide per min. Specific activity was expressed as units per mg of protein. For experiments related to Kₘ determination of purified enzymes, various concentrations of ATP or IκBα peptide were used in the assay at either a fixed IκBα or ATP concentration. For IκBα peptide Kₘ, assays were carried out with 0.1 μg of enzyme, 5 μM ATP and IκBα peptide from 0.5 to 20 μM. For ATP Kₘ, assays were carried out with 0.1 μg of enzyme, 10 μM IκBα peptide and ATP from 0.1 to 10 μM. For Kₘ determination of rhIKK1 homodimer, due to its low activity and higher Kₘ for IκBα peptide, rhIKK1 homodimer (0.3 μg) was assayed with 125 μM IκBα peptide and a 5-fold higher specific activity of ATP (from 0.1 to 10 μM) for ATP Kₘ experiments and a 5-fold higher specific activity of 5 μM ATP and IκBα peptide (from 5 to 200 μM) for IκBα peptide Kₘ experiments.

IKKβ Resin Enzyme Assay

IKKβ kinase activity was measured using a biotinylated IκBα peptide (Gly-Leu-Lys-Lys-Glu-Arg-Leu-Leu-Asp-Asp-Arg-His-Asp-Ser₃₂-Gly-Leu-Asp-Ser₃₆-Met-Lys-Asp-Glu-Glu) (American Peptide Co.). 20 ul of the standard reaction mixture contained 5 μM biotinylated IκBα peptide, 0.1 μCi/reaction [γ-³³P] ATP (Amersham) (about 1×10⁵ cpm), 1 μM ATP (Sigma), 1 mM DTT (Sigma), 2 mM MgCl₂ (Sigma), 2 mM MnCl₂ (Sigma), 10 mM NaF (Sigma), 25 mM Hepes (Sigma) buffer, pH 7.6 and 20 μl enzyme solution and 10 ul inhibitor in a final volume of 50 μl. After incubation at 25° C. for 30 min, 150 μl resin (Dowex anion-exchange resin AG1X8 200-400 mesh) in 900 mM formate, pH 3.0 was added to each well to stop the reaction. Resin was allowed to settle for one hour and 50 μl of supernatant was removed to a Micolite-2 flat bottom plate (Dynex). 150 μl of scintillation fluid (Microscint 40) (Packard) was added to each well. Incorporation of [γ-³³P] ATP was measured using a Top-Count NXT (Packard).

IKK heterodimer Resin Enzyme Assay

IKK heterodimer kinase activity was measured using a biotinylated IκBα peptide (Gly-Leu-Lys-Lys-Glu-Arg-Leu-Leu-Asp-Asp-Arg-His-Asp-Ser₃₂-Gly-Leu-Asp-Ser₃₆-Met-Lys-Asp-Glu-Glu) (American Peptide Co.). 20 ul of the standard reaction mixture contained 5 μM biotinylated IκBα peptide, 0.1 μCi/reaction [γ-³³P] ATP (Amersham) (about 1×10⁵ cpm), 1 μM ATP (Sigma), 1 mM DTT (Sigma), 2 mM MgCl₂ (Sigma), 2 mM MnCl₂ (Sigma), 10 mM NaF (Sigma), 25 mM Hepes (Sigma) buffer, pH 7.6 and 20 μl enzyme solution and 10 μl inhibitor in a final volume of 50 μl. After incubation at 25° C. for 30 min, 150 μl resin (Dowex anion-exchange resin AG1X8 200-400 mesh) in 900 mM formate, pH 3.0 was added to each well to stop the reaction. Resin was allowed to settle for one hour and 50 μl of supernatant was removed to a Micolite-2 flat bottom plate (Dynex). 150 μl of scintillation fluid (Microscint 40) (Packard) was added to each well. Incorporation of [γ-³³P] ATP was measured using a Top-Count NXT (Packard).

What is claimed is:

1. A compound of formula I 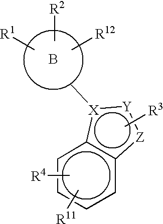
wherein
B is a 5 or 6 membered heteroaryl, aryl, saturated or unsaturated heterocyclic wherein said aryl, heteroaryl, or heterocyclic are optionally substituted with R¹, R², and R²;
X is selected from the group consisting of: N and C;
Y and Z are independently selected from the group consisting of: N, CH, CR³, S, and O;
R¹ is selected from the group consisting of: hydrido, halogen, alkyl, aryl, heteroaryl, alkenyl, alkynyl, haloalkyl, CN, NO₂, OR⁵, OCOOR⁵, CO₂R⁷, CON(R⁶)R⁷, COR⁶, SR⁶, SOR⁶, SO₂R⁶, NR⁶R⁷, NR⁶COR⁷, NR⁶CONHR⁷, NR⁶SO₂R⁷, NR⁶SO₂NHR⁷, and SO₂N(R⁶)R⁷ wherein R⁶ and R⁷ may be taken together to form a 3-7 membered carbocyclic ring having 1 to 3 substituted or unsubstituted heteroatoms selected from the group consisting of: S, SO, SO₂, O, and NR⁶; wherein said alkenyl, alkynyl, alkyl, aryl, heteroaryl or OR⁵ are optional substituted with, hydrido, halogen, alkyl, hydroxyalkyl, aryl, heteroaryl, haloalkyl, COCF₃, CN, NO₂, OR⁵, OCOOR⁵, CO₂R⁷, CON(R⁶)R⁷ COR⁶, SR⁶, SOR⁶, SO₂R⁶, NR⁶R⁷, NR⁶COR⁷, NR⁶CONHR⁷, NR⁶SO₂R⁷, NR⁶SO₂NHR⁷, and SO₂N(R⁶)R⁷ wherein R⁶ and R⁷ may be taken together to form a 3-7 membered carbocyclic ring having 1 to 3 substituted or unsubstituted heteroatoms selected from the group consisting of: S, SO, SO₂, O, and NR⁶;
R² is selected from the group consisting of: halogen, hydrido, hydroxyalkyl, alkyl, OR⁶, CN, NO₂, SR⁶, NHR⁶, CON(R⁶)R⁷, NHCONHR⁶, CO₂H, and haloalkyl;
R¹ and R² may be taken together to form a 5 to 7 membered saturated or unsaturated carbocyclic ring optionally containing 0 to 3 heteroatoms selected from the group consisting of N, O, or S, and wherein said ring is optionally substituted with R^{l};
R³ is selected from the group consisting of: substituted or unsubstituted amidine, alkylamino, aminoalkyl, CONHR⁷, NH₂, NHCOR⁶, and CH₂NHCOR⁶;
R⁴ is selected from the group consisting of: halogen, alkylsulfinyl, alkylsulfonyl, cyano, alkoxycarbonyl, alkyl, haloalkyl, hydrido, hydroxyalkyl, haloalkoxy, heterocyclic, nitro, acylamino, aryl, heteroaryl, and alkenyl, OR¹³, SR⁸, SO₂N(R⁸)R^{8′}, NHR⁹, NHCOR⁹, NR⁹COR⁹, NHCO(OR⁹), NR⁹CO(OR⁹), NR⁸SO₂R¹⁰, NHSO₂N(R¹⁰)R^{10′}, NR⁶CON(R¹⁰)R^{10′}, COR⁹, CO₂R⁸, CON(R⁸)R^{8′}, wherein R⁸ and R^{8′} may be taken together to form a 3-7 membered carbocyclic ring having 1 to 3 substituted or unsubstituted heteroatoms selected from S, SO, SO₂, O, N, and NR⁶, and wherein R¹⁰ and R^{10′} may be taken together to form a 3-7 membered carbocyclic ring having 1 to 3 substituted or unsubstituted heteroatoms selected from S, SO, SO₂, O, N, and NR⁶ wherein said aryl, heterocyclic, heteroaryl, or alkenyl are optionally substituted with R⁹;
R⁵ is selected from the group consisting of: hydrido, alkyl, aryl, arylalkyl, heteroaryl, heterocyclicalkyl, and heteroarylalkyl, wherein aryl, alkyl, arylalkyl, heteroaryl, heterocyclicalkyl, or heteroarylalkyl are optionally substituted with one or more radicals selected from the group consisting of OR¹⁴, N(R¹⁴)R^{14′}, and glycols;
R⁶ is independently selected from the group consisting of: hydrido, aryl, heteroaryl, lower alkyl, haloalkyl, alkenyl, alkynyl, hydroxyalkyl, aminoalkyl, alkylaminoalkyl, alkoxy, alkoxyalkyl, heterocyclicalkyl, and heterocyclic;
R⁷ is independently selected from the group consisting of: hydrido, aryl, heteroaryl, lower alkyl, haloalkyl, alkenyl, alkynyl, hydroxyalkyl, aminoalkyl, alkylaminoalkyl, alkoxy, alkoxyalkyl, heterocyclicalkyl, and heterocyclic;
R⁸ is independently selected from the group consisting of: hydrido, aryl, heteroaryl, arylalkyl, heterocyclic, haloalkyl, arylalkylamino, alkylaminoalkyl, dialkylaminoalkyl, alkyl, alkenyl, alkynyl, heteroarylalkyl, and heterocyclicalkyl;
R^{8′} is independently selected from the group consisting of: hydrido, aryl, heteroaryl, arylalkyl, heterocyclic, haloalkyl, arylalkylamino, alkylaminoalkyl, dialkylaminoalkyl, alkyl, alkenyl, alkynyl, heteroarylalkyl, and heterocyclicalkyl;
R⁹ is independently selected from the group consisting of: hydrido, lower alkyl, aryl, heteroaryl, arylalkyl, heterocyclic, cycloalkyl, heterocyclicalkyl, haloalkyl, arylalkylamino, amino, aminoalkyl, aminoacyl, nitro, azido, and heteroarylalkyl, wherein alkyl, aryl, heteroaryl, aminoalkyl, or arylalkyl are optionally substituted with one or more radical selected from the group consisting of: alkylsulfonamide, sulfamyl, alkyl, alkylthio, alkylsulfinyl, alkylsulfonyl, alkylamino, aminoalkyl, alkylaminoalkyl, alkoxy, halogen, acyloxy, oxy, formyl, haloalkyl, cyano, haloalkoxy, acyl, carboxyl, hydroxy, hydroxyalkyloxy, phenoxy, nitro, azido, benzyloxy, dialkylaminoacyl, thioalkyl, aminoacyloxy, thiocyanate, isothiocyanate, alkyldioxy, hydroxyalkyl, alkylamino, alkyloxycarbonyl, alkoxyalkyl, alkenylamino, alkynylamino, alkenyl, alkynyl, dialkylaminoalkyloxy, and heterocyclic optionally substituted with alkyl, alkylamino, aminoalkyl, and alkylaminoalkyl;
R¹⁰ is independently selected from the group consisting of: hydrido, lower alkyl, heteroaryl, heterocyclic, haloalkyl, arylalkylamino, heteroarylalkyl, aryl, and arylalkyl, wherein aryl, heteroaryl, heterocyclic, or arylalkyl are optionally substituted with one or more radical selected from alkyl, alkoxy, halogen, haloalkyl, cyano, haloalkoxy, acyl, carboxyl, hydroxy, hydroxyalkyloxy, phenoxy, benzyloxy, dialkylaminoalkyloxy, and heterocyclic,
R^{10′} is independently selected from the group consisting of: hydrido, lower alkyl, heteroaryl, heterocyclic, haloalkyl, arylalkylamino, heteroarylalkyl, aryl, and arylalkyl, wherein aryl, heteroaryl, heterocyclic, or arylalkyl are optionally substituted with one or more radical selected from alkyl, alkoxy, halogen, haloalkyl, cyano, haloalkoxy, acyl, carboxyl, hydroxy, hydroxyalkyloxy, phenoxy, benzyloxy, dialkylaminoalkyloxy, and heterocyclic,
R¹¹ is selected from the group consisting of: hydrido, halogen, haloalkyl, CN, CO₂R⁵, lower alkyl, lower alkenyl, lower alkynyl, alkoxy, and CONH₂;
R¹² is selected from the group consisting of: hydrido, halogen, alkyl, and alkoxy;
R¹³ is selected from the group consisting of: hydrido, alkyl, aryl, arylalkyl, heteroaryl, heterocyclicalkyl, and heteroarylalkyl, wherein aryl, alkyl, arylalkyl, heteroaryl, heterocyclicalkyl, or heteroarylalkyl are optionally substituted with one or more radicals selected from the group consisting of OR¹⁴, N(R¹⁴)R^{14′}, and glycols;
R¹⁴ is independently selected from the group consisting of hydrido, and lower alkyl; and
R^{14′} is independently selected from the group consisting of hydrido, and lower alkyl; or isomers, tautomers, carriers, esters, prodrugs, pharmaceutically acceptable salts thereof.

2. A compound of formula II 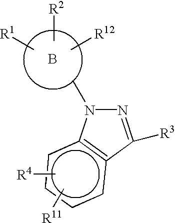
wherein
B is a 5 or 6 membered heteroaryl, aryl, saturated or unsaturated heterocyclic wherein said aryl, heteroaryl, or heterocyclic are optionally substituted with R¹, R², and R¹²;
R¹ is selected from the group consisting of: hydrido, halogen, alkyl, aryl, heteroaryl, alkenyl, alkynyl, haloalkyl, CN, NO₂, OR⁵, OCOOR⁵, CO₂R⁷, CON(R⁶)R⁷, COR⁶, SR⁶, SOR⁶, SO₂R⁶, NR⁶R⁷, NR⁶COR⁷, NR⁶CONHR⁷, NR⁶SO₂R⁷, NR⁶SO₂NHR⁷, and SO₂N(R⁶)R⁷ wherein R⁶ and R⁷may be taken together to form a 3-7 membered carbocyclic ring having 1 to 3 substituted or unsubstituted heteroatoms selected from the group consisting of: S, SO, SO₂, O, and NR⁶; wherein said alkenyl, alkynyl, alkyl, aryl, heteroaryl or OR⁵ are optional substituted with, hydrido, halogen, alkyl, hydroxyalkyl, aryl, heteroaryl, haloalkyl, COCF₃, CN, NO₂, OR⁵, OCOOR⁵, CO₂R⁷, CON(R⁶)R⁷, COR⁶, SR⁶, SOR⁶, SO₂R⁶, NR⁶R⁷, NR⁶COR⁷, NR⁶CONHR⁷, NR⁶SO₂R⁷, NR⁶SO₂NHR⁷, and SO₂N(R⁶)R⁷ wherein R⁶ and R⁷ may be taken together to form a 3-7 membered carbocyclic ring having 1 to 3 substituted or unsubstituted heteroatoms selected from the group consisting of: S, SO, SO₂, O, and NR⁶;
R² is selected from the group consisting of: halogen, hydrido, hydroxyalkyl, alkyl, OR⁶, CN, NO₂, SR⁶, NHR⁶, CON(R⁶)R⁷, NHCONHR⁶, CO₂H, and haloalkyl;
R¹ and R² may be taken together to form a 5 to 7 membered saturated or unsaturated carbocyclic ring optionally containing 0 to 3 heteroatoms selected from the group consisting of N, O, or S, and wherein said ring is optionally substituted with R¹;
R³ is selected from the group consisting of: substituted or unsubstituted amidine, alkylamino, aminoalkyl, CONHR⁷, NH₂, NHCOR⁶, and CH₂NHCOR⁶;
R⁴ is selected from the group consisting of: halogen, alkylsulfinyl, alkylsulfonyl, cyano, alkoxycarbonyl, alkyl, haloalkyl, hydrido, hydroxyalkyl, haloalkoxy, heterocyclic, nitro, acylamino, aryl, heteroaryl, and alkenyl, OR¹³, SR⁸, SO₂N(R⁸)R^{8′}, NHR⁹, NHCOR⁹, NR⁹COR⁹, NHCO(OR⁹), NR⁹CO(OR⁹), NR⁸SO₂R¹⁰, NHSO₂N(R¹⁰)R^{10′}, NR⁶CON(R¹⁰)R^{10′}, COR⁹, CO₂R⁸, CON(R⁸)R^{8′}, wherein R⁸ and R^{8′} may be taken together to form a 3-7 membered carbocyclic ring having 1 to 3 substituted or unsubstituted heteroatoms selected from S, SO, SO₂, O, N, and NR⁶, and wherein R¹⁰ and R^{10′} may be taken together to form a 3-7 membered carbocyclic ring having 1 to 3 substituted or unsubstituted heteroatoms selected from S, SO, SO₂, O, N, and NR⁶ wherein said aryl, heterocyclic, heteroaryl, or alkenyl are optionally substituted with R⁹;
R⁵ is selected from the group consisting of: hydrido, alkyl, aryl, arylalkyl, heteroaryl, heterocyclicalkyl, and heteroarylalkyl, wherein aryl, alkyl, arylalkyl, heteroaryl, heterocyclicalkyl, or heteroarylalkyl are optionally substituted with one or more radicals selected from the group consisting of OR¹⁴, N(R¹⁴)R^{14′}, and glycols;
R⁶ is independently selected from the group consisting of: hydrido, aryl, heteroaryl, lower alkyl, haloalkyl, alkenyl, alkynyl, hydroxyalkyl, aminoalkyl, alkylaminoalkyl, alkoxy, alkoxyalkyl, heterocyclicalkyl, and heterocyclic;
R⁷ is independently selected from the group consisting of: hydrido, aryl, heteroaryl, lower alkyl, haloalkyl, alkenyl, alkynyl, hydroxyalkyl, aminoalkyl, alkylaminoalkyl, alkoxy, alkoxyalkyl, heterocyclicalkyl, and heterocyclic;
R⁸ is independently selected from the group consisting of: hydrido, aryl, heteroaryl, arylalkyl, heterocyclic, haloalkyl, arylalkylamino, alkylaminoalkyl, dialkylaminoalkyl, alkyl, alkenyl, alkynyl, heteroarylalkyl, and heterocyclicalkyl;
R^{8′} is independently selected from the group consisting of: hydrido, aryl, heteroaryl, arylalkyl, heterocyclic, haloalkyl, arylalkylamino, alkylaminoalkyl, dialkylaminoalkyl, alkyl, alkenyl, alkynyl, heteroarylalkyl, and heterocyclicalkyl;
R⁹ is independently selected from the group consisting of: hydrido, lower alkyl, aryl, heteroaryl, arylalkyl, heterocyclic, cycloalkyl, heterocyclicalkyl, haloalkyl, arylalkylamino, amino, aminoalkyl, aminoacyl, nitro, azido, and heteroarylalkyl, wherein alkyl, aryl, heteroaryl, aminoalkyl, or arylalkyl are optionally substituted with one or more radical selected from the group consisting of: alkylsulfonamide, sulfamyl, alkyl, alkylthio, alkylsulfinyl, alkylsulfonyl, alkylamino, aminoalkyl, alkylaminoalkyl, alkoxy, halogen, acyloxy, oxy, formyl, haloalkyl, cyano, haloalkoxy, acyl, carboxyl, hydroxy, hydroxyalkyloxy, phenoxy, nitro, azido, benzyloxy, dialkylaminoacyl, thioalkyl, aminoacyloxy, thiocyanate, isothiocyanate, alkyldioxy, hydroxyalkyl, alkylamino, alkyloxycarbonyl, alkoxyalkyl, alkenylamino, alkynylamino, alkenyl, alkynyl, dialkylaminoalkyloxy, and heterocyclic optionally substituted with alkyl, alkylamino, aminoalkyl, and alkylaminoalkyl;
R¹⁰ is independently selected from the group consisting of: hydrido, lower alkyl, heteroaryl, heterocyclic, haloalkyl, arylalkylamino, heteroarylalkyl, aryl, and arylalkyl, wherein aryl, heteroaryl, heterocyclic, or arylalkyl are optionally substituted with one or more radical selected from alkyl, alkoxy, halogen, haloalkyl, cyano, haloalkoxy, acyl, carboxyl, hydroxy, hydroxyalkyloxy, phenoxy, benzyloxy, dialkylaminoalkyloxy, and heterocyclic,
R^{10′} is independently selected from the group consisting of: hydrido, lower alkyl, heteroaryl, heterocyclic, haloalkyl, arylalkylamino, heteroarylalkyl, aryl, and arylalkyl, wherein aryl, heteroaryl, heterocyclic, or arylalkyl are optionally substituted with one or more radical selected from alkyl, alkoxy, halogen, haloalkyl, cyano, haloalkoxy, acyl, carboxyl, hydroxy, hydroxyalkyloxy, phenoxy, benzyloxy, dialkylaminoalkyloxy, and heterocyclic,
R¹¹ is selected from the group consisting of: hydrido, halogen, haloalkyl, CN, CO₂R⁵, lower alkyl, lower alkenyl, lower alkynyl, alkoxy, and CONH₂;
R¹² is selected from the group consisting of: hydrido, halogen, alkyl, and alkoxy;
R¹³ is selected from the group consisting of: hydrido, alkyl, aryl, arylalkyl, heteroaryl, heterocyclicalkyl, and heteroarylalkyl, wherein aryl, alkyl, arylalkyl, heteroaryl, heterocyclicalkyl, or heteroarylalkyl are optionally substituted with one or more radicals selected from the group consisting of OR¹⁴, N(R¹⁴)R^{14′}, and glycols;
R¹⁴ is independently selected from the group consisting of hydrido, and lower alkyl; and
R^{14′} is independently selected from the group consisting of hydrido, and lower alkyl;
or isomers, tautomers, carriers, esters, prodrugs, pharmaceutically acceptable salts thereof.

3. The compound of claim 2 of formula II 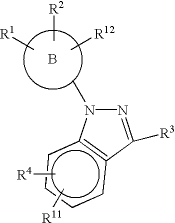
wherein
B is a 5 or 6 membered heteroaryl, aryl, saturated or unsaturated heterocyclic wherein said aryl, heteroaryl, or heterocyclic are optionally substituted with R¹, R², and R¹²;
R¹ is selected from the group consisting of: hydrido, halogen, alkyl, aryl, heteroaryl, alkenyl, alkynyl, haloalkyl, CN, NO₂, OR⁵, OCOOR⁵, CO₂R⁷, CON(R⁶)R⁷, COR⁶, SR⁶, SOR⁶, SO₂R⁶, NR⁶R⁷, NR⁶COR⁷, NR⁶CONHR⁷, NR⁶SO₂R⁷, NR⁶SO₂NHR⁷, and SO₂N(R⁶)R⁷ wherein R⁶ and R⁷ may be taken together to form a 3-7 membered carbocyclic ring having 1 to 3 substituted or unsubstituted heteroatoms selected from the group consisting of: S, SO, SO₂, O, and NR⁶; wherein said alkenyl, alkynyl, alkyl, aryl, heteroaryl or OR⁵ are optional substituted with, hydrido, halogen, alkyl, hydroxyalkyl, aryl, heteroaryl, haloalkyl, COCF₃, CN, NO₂, OR⁵, OCOOR⁵, CO₂R⁷, CON(R⁶)R⁷, COR⁶, SR⁶, SOR⁶, SO₂R⁶, NR⁶R⁷, NR⁶COR⁷, NR⁶CONHR⁷, NR⁶SO₂R⁷, NR⁶SO₂NHR⁷, and SO₂N(R⁶)R⁷ wherein R⁶ and R⁷ may be taken together to form a 3-7 membered carbocyclic ring having 1 to 3 substituted or unsubstituted heteroatoms selected from the group consisting of: S, SO, SO₂, O, and NR⁶;
R² is selected from the group consisting of: halogen, hydrido, hydroxyalkyl, alkyl, OR⁶, CN, NO₂, SR⁶, NHR⁶, CON(R⁶)R⁷, NHCONHR⁶, CO₂H, and haloalkyl;
R¹ and R² may be taken together to form a 5 to 7 membered saturated or unsaturated carbocyclic ring optionally containing 0 to 3 heteroatoms selected from the group consisting of N, O, or S, and wherein said ring is optionally substituted with R¹;
R³ is selected from the group consisting of: substituted or unsubstituted amidine, alkylamino, aminoalkyl, CONHR⁷, NH₂, NHCOR⁶, and CH₂NHCOR⁶;
R⁴ is selected from the group consisting of: halogen, alkylsulfinyl, alkylsulfonyl, cyano, alkoxycarbonyl, alkyl, haloalkyl, hydrido, hydroxyalkyl, haloalkoxy, heterocyclic, nitro, acylamino, aryl, heteroaryl, and alkenyl, OR¹³, SR⁸, SO₂N(R⁸)R^{8′}, NHR⁹, NHCOR⁹, NR⁹COR⁹, NHCO(OR⁹), NR⁹CO(OR⁹), NR⁸SO₂R¹⁰, NHSO₂N(R¹⁰)R^{10′}, NR⁶CON(R¹⁰)R^{10′}, COR⁹, CO₂R⁸, CON(R⁸)R^{8′}, wherein R⁸ and R^{8′} may be taken together to form a 3-7 membered carbocyclic ring having 1 to 3 substituted or unsubstituted heteroatoms selected from S, SO, SO₂, O, N, and NR⁶, and wherein R¹⁰ and R^{10′} may be taken together to form a 3-7 membered carbocyclic ring having 1 to 3 substituted or unsubstituted heteroatoms selected from S, SO, SO₂, O, N, and NR⁶ wherein said aryl, heterocyclic, heteroaryl, or alkenyl are optionally substituted with R⁹;
R⁵ is selected from the group consisting of: hydrido, alkyl, aryl, arylalkyl, heteroaryl, heterocyclicalkyl, and heteroarylalkyl, wherein aryl, alkyl, arylalkyl, heteroaryl, heterocyclicalkyl, or heteroarylalkyl are optionally substituted with one or more radicals selected from the group consisting of OR¹⁴, N(R¹⁴)R^{14′}, and glycols;
R⁶ is independently selected from the group consisting of: hydrido, aryl, heteroaryl, lower alkyl, haloalkyl, alkenyl, alkynyl, hydroxyalkyl, aminoalkyl, alkylaminoalkyl, alkoxy, alkoxyalkyl, heterocyclicalkyl, and heterocyclic;
R⁷ is independently selected from the group consisting of: hydrido, aryl, heteroaryl, lower alkyl, haloalkyl, alkenyl, alkynyl, hydroxyalkyl, aminoalkyl, alkylaminoalkyl, alkoxy, alkoxyalkyl, heterocyclicalkyl, and heterocyclic;
R⁸ is independently selected from the group consisting of: hydrido, aryl, heteroaryl, arylalkyl, heterocyclic, haloalkyl, arylalkylamino, alkylaminoalkyl, dialkylaminoalkyl, alkyl, alkenyl, alkynyl, heteroarylalkyl, and heterocyclicalkyl;
R^{8′} is independently selected from the group consisting of: hydrido, aryl, heteroaryl, arylalkyl, heterocyclic, haloalkyl, arylalkylamino, alkylaminoalkyl, dialkylaminoalkyl, alkyl, alkenyl, alkynyl, heteroarylalkyl, and heterocyclicalkyl;
R⁹ is independently selected from the group consisting of: hydrido, lower alkyl, aryl, heteroaryl, arylalkyl, heterocyclic, cycloalkyl, heterocyclicalkyl, haloalkyl, arylalkylamino, amino, aminoalkyl, aminoacyl, nitro, azido, and heteroarylalkyl, wherein alkyl, aryl, heteroaryl, aminoalkyl, or arylalkyl are optionally substituted with one or more radical selected from the group consisting of: alkylsulfonamide, sulfamyl, alkyl, alkylthio, alkylsulfinyl, alkylsulfonyl, alkylamino, aminoalkyl, alkylaminoalkyl, alkoxy, halogen, acyloxy, oxy, formyl, haloalkyl, cyano, haloalkoxy, acyl, carboxyl, hydroxy, hydroxyalkyloxy, phenoxy, nitro, azido, benzyloxy, dialkylaminoacyl, thioalkyl, aminoacyloxy, thiocyanate, isothiocyanate, alkyldioxy, hydroxyalkyl, alkylamino, alkyloxycarbonyl, alkoxyalkyl, alkenylamino, alkynylamino, alkenyl, alkynyl, dialkylaminoalkyloxy, and heterocyclic optionally substituted with alkyl, alkylamino, aminoalkyl, and alkylaminoalkyl;
R¹⁰ is independently selected from the group consisting of: hydrido, lower alkyl, heteroaryl, heterocyclic, haloalkyl, arylalkylamino, heteroarylalkyl, aryl, and arylalkyl, wherein aryl, heteroaryl, heterocyclic, or arylalkyl are optionally substituted with one or more radical selected from alkyl, alkoxy, halogen, haloalkyl, cyano, haloalkoxy, acyl, carboxyl, hydroxy, hydroxyalkyloxy, phenoxy, benzyloxy, dialkylaminoalkyloxy, and heterocyclic,
R^{10′} is independently selected from the group consisting of: hydrido, lower alkyl, heteroaryl, heterocyclic, haloalkyl, arylalkylamino, heteroarylalkyl, aryl, and arylalkyl, wherein aryl, heteroaryl, heterocyclic, or arylalkyl are optionally substituted with one or more radical selected from alkyl, alkoxy, halogen, haloalkyl, cyano, haloalkoxy, acyl, carboxyl, hydroxy, hydroxyalkyloxy, phenoxy, benzyloxy, dialkylaminoalkyloxy, and heterocyclic,
R¹¹ is hydrido;
R¹² is hydrido;
R¹³ is selected from the group consisting of: hydrido, alkyl, aryl, arylalkyl, heteroaryl, heterocyclicalkyl, and heteroarylalkyl, wherein aryl, alkyl, arylalkyl, heteroaryl, heterocyclicalkyl, or heteroarylalkyl are optionally substituted with one or more radicals selected from the group consisting of OR¹⁴, N(R¹⁴)R^{14′}, and glycols;
R¹⁴ is independently selected from the group consisting of hydrido, and lower alkyl; and
R^{14′} is independently selected from the group consisting of hydrido, and lower alkyl;
or isomers, tautomers, carriers, esters, prodrugs, pharmaceutically acceptable salts thereof.

4. The compound of claim 2 of formula II 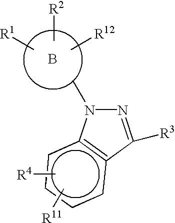
wherein
B is a 5 or 6 membered heteroaryl, aryl, saturated or unsaturated heterocyclic wherein said aryl, heteroaryl, or heterocyclic are optionally substituted with R¹, R², and R¹²;
R¹ is selected from the group consisting of: hydrido, halogen, alkyl, aryl, heteroaryl, alkenyl, alkynyl, haloalkyl, CN, NO₂, OR⁵, OCOOR⁵, CO₂R⁷, CON(R⁶)R⁷, COR⁶, SR⁶, SOR⁶, SO₂R⁶, NR⁶R⁷, NR⁶COR⁷, NR⁶CONHR⁷, NR⁶SO₂R⁷, NR⁶SO₂NHR⁷, and SO₂N(R⁶)R⁷ wherein R⁶ and R⁷ may be taken together to form a 3-7 membered carbocyclic ring having 1 to 3 substituted or unsubstituted heteroatoms selected from the group consisting of: S, SO, SO₂, O, and NR⁶; wherein said alkenyl, alkynyl, alkyl, aryl, heteroaryl or OR⁵ are optional substituted with, hydrido, halogen, alkyl, hydroxyalkyl, aryl, heteroaryl, haloalkyl, COCF₃, CN, NO₂, OR⁵, OCOOR⁵, CO₂R⁷, CON(R⁶)R⁷, COR⁶, SR⁶, SOR⁶, SO₂R⁶, NR⁶R⁷, NR⁶COR⁷, NR⁶CONHR⁷, NR⁶SO₂R⁷, NR⁶SO₂NHR⁷, and SO₂N(R⁶)R⁷ wherein R⁶ and R⁷ may be taken together to form a 3-7 membered carbocyclic ring having 1 to 3 substituted or unsubstituted heteroatoms selected from the group consisting of: S, SO, SO₂, O, and NR⁶;
R² is hydrido;
R³ is selected from the group consisting of: substituted or unsubstituted amidine, alkylamino, aminoalkyl, CONHR⁷, NH₂, NHCOR⁶, and CH₂NHCOR⁶;
R⁴ is selected from the group consisting of: halogen, alkylsulfinyl, alkylsulfonyl, cyano, alkoxycarbonyl, alkyl, haloalkyl, hydrido, hydroxyalkyl, haloalkoxy, heterocyclic, nitro, acylamino, aryl, heteroaryl, and alkenyl, OR¹³, SR⁸, SO₂N(R⁸)R^{8′}, NHR⁹, NHCOR⁹, NR⁹COR⁹, NHCO(OR⁹), NR⁹CO(OR⁹), NR⁸SO₂R¹⁰, NHSO₂N(¹⁰)R^{10′}, NR⁶CON(R¹⁰)R^{10′}, COR⁹, CO₂R⁸, CON(R⁸)R^{8′}, wherein R⁸ and R^{8′} may be taken together to form a 3-7 membered carbocyclic ring having 1 to 3 substituted or unsubstituted heteroatoms selected from S, SO, SO₂, O, N, and NR⁶, and wherein R¹⁰ and R^{10′} may be taken together to form a 3-7 membered carbocyclic ring having 1 to 3 substituted or unsubstituted heteroatoms selected from S, SO, SO₂, O, N, and NR⁶ wherein said aryl, heterocyclic, heteroaryl, or alkenyl are optionally substituted with R⁹;
R⁵ is selected from the group consisting of: hydrido, alkyl, aryl, arylalkyl, heteroaryl, heterocyclicalkyl, and heteroarylalkyl, wherein aryl, alkyl, arylalkyl, heteroaryl, heterocyclicalkyl, or heteroarylalkyl are optionally substituted with one or more radicals selected from the group consisting of OR¹⁴, N(R¹⁴)R^{14′}, and glycols;
R⁶ is independently selected from the group consisting of: hydrido, aryl, heteroaryl, lower alkyl, haloalkyl, alkenyl, alkynyl, hydroxyalkyl, aminoalkyl, alkylaminoalkyl, alkoxy, alkoxyalkyl, heterocyclicalkyl, and heterocyclic;
R⁷ is independently selected from the group consisting of: hydrido, aryl, heteroaryl, lower alkyl, haloalkyl, alkenyl, alkynyl, hydroxyalkyl, aminoalkyl, alkylaminoalkyl, alkoxy, alkoxyalkyl, heterocyclicalkyl, and heterocyclic;
R⁸ is independently selected from the group consisting of: hydrido, aryl, heteroaryl, arylalkyl, heterocyclic, haloalkyl, arylalkylamino, alkylaminoalkyl, dialkylaminoalkyl, alkyl, alkenyl, alkynyl, heteroarylalkyl, and heterocyclicalkyl;
R^{8′} is independently selected from the group consisting of: hydrido, aryl, heteroaryl, arylalkyl, heterocyclic, haloalkyl, arylalkylamino, alkylaminoalkyl, dialkylaminoalkyl, alkyl, alkenyl, alkynyl, heteroarylalkyl, and heterocyclicalkyl;
R⁹ is independently selected from the group consisting of: hydrido, lower alkyl, aryl, heteroaryl, arylalkyl, heterocyclic, cycloalkyl, heterocyclicalkyl, haloalkyl, arylalkylamino, amino, aminoalkyl, aminoacyl, nitro, azido, and heteroarylalkyl, wherein alkyl, aryl, heteroaryl, aminoalkyl, or arylalkyl are optionally substituted with one or more radical selected from the group consisting of: alkylsulfonamide, sulfamyl, alkyl, alkylthio, alkylsulfinyl, alkylsulfonyl, alkylamino, aminoalkyl, alkylaminoalkyl, alkoxy, halogen, acyloxy, oxy, formyl, haloalkyl, cyano, haloalkoxy, acyl, carboxyl, hydroxy, hydroxyalkyloxy, phenoxy, nitro, azido, benzyloxy, dialkylaminoacyl, thioalkyl, aminoacyloxy, thiocyanate, isothiocyanate, alkyldioxy, hydroxyalkyl, alkylamino, alkyloxycarbonyl, alkoxyalkyl, alkenylamino, alkynylamino, alkenyl, alkynyl, dialkylaminoalkyloxy, and heterocyclic optionally substituted with alkyl, alkylamino, aminoalkyl, and alkylaminoalkyl;
R¹⁰ is independently selected from the group consisting of: hydrido, lower alkyl, heteroaryl, heterocyclic, haloalkyl, arylalkylamino, heteroarylalkyl, aryl, and arylalkyl, wherein aryl, heteroaryl, heterocyclic, or arylalkyl are optionally substituted with one or more radical selected from alkyl, alkoxy, halogen, haloalkyl, cyano, haloalkoxy, acyl, carboxyl, hydroxy, hydroxyalkyloxy, phenoxy, benzyloxy, dialkylaminoalkyloxy, and heterocyclic,
R^{10′} is independently selected from the group consisting of: hydrido, lower alkyl, heteroaryl, heterocyclic, haloalkyl, arylalkylamino, heteroarylalkyl, aryl, and arylalkyl, wherein aryl, heteroaryl, heterocyclic, or arylalkyl are optionally substituted with one or more radical selected from alkyl, alkoxy, halogen, haloalkyl, cyano, haloalkoxy, acyl, carboxyl, hydroxy, hydroxyalkyloxy, phenoxy, benzyloxy, dialkylaminoalkyloxy, and heterocyclic,
R¹¹ is hydrido;
R¹² is hydrido;
R¹³ is selected from the group consisting of: hydrido, alkyl, aryl, arylalkyl, heteroaryl, heterocyclicalkyl, and heteroarylalkyl, wherein aryl, alkyl, arylalkyl, heteroaryl, heterocyclicalkyl, or heteroarylalkyl are optionally substituted with one or more radicals selected from the group consisting of OR¹⁴, N(R¹⁴)R^{14′}, and glycols;
R¹⁴ is independently selected from the group consisting of hydrido, and lower alkyl; and
R^{14′} is independently selected from the group consisting of hydrido, and lower alkyl;
or isomers, tautomers, carriers, esters, prodrugs, pharmaceutically acceptable salts thereof.

5. The compound of claim 2 of formula II 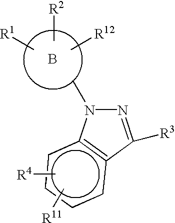
wherein
B is a 5 or 6 membered heteroaryl, aryl, saturated or unsaturated heterocyclic wherein said aryl, heteroaryl, or heterocyclic are optionally substituted with R¹, R², and R¹²;
R¹ is selected from the group consisting of: hydrido, halogen, alkyl, aryl, heteroaryl, alkenyl, alkynyl, haloalkyl, CN, NO₂, OR⁵, OCOOR⁵, CO₂R⁷, CON(R⁶)R⁷, COR⁶, SR⁶, SOR⁶, SO₂R⁶, NR⁶R⁷, NR⁶COR⁷, NR⁶CONHR⁷, NR⁶SO₂R⁷, NR⁶SO₂NHR⁷, and SO₂N(R⁶)R⁷ wherein R⁶ and R⁷ may be taken together to form a 3-7 membered carbocyclic ring having 1 to 3 substituted or unsubstituted heteroatoms selected from the group consisting of: S, SO, SO₂, O, and NR⁶; wherein said alkenyl, alkynyl, alkyl, aryl, heteroaryl or OR⁵ are optional substituted with, hydrido, halogen, alkyl, hydroxyalkyl, aryl, heteroaryl, haloalkyl, COCF₃, CN, NO₂, OR⁵, OCOOR⁵, CO₂R⁷, CON(R⁶)R⁷, COR⁶, SR⁶, SOR⁶, SO₂R⁶, NR⁶R⁷, NR⁶COR⁷, NR⁶CONHR⁷, NR⁶SO₂R⁷, NR⁶SO₂NHR⁷, and SO₂N(R⁶)R⁷ wherein R⁶ and R⁷ may be taken together to form a 3-7 membered carbocyclic ring having 1 to 3 substituted or unsubstituted heteroatoms selected from the group consisting of: S, SO, SO₂, O, and NR⁶;
R² is selected from the group consisting of: halogen, hydrido, hydroxyalkyl, alkyl, OR⁶, CN, NO₂, SR⁶, NHR⁶, CON(R⁶)R⁷, NHCONHR⁶, CO₂H, and haloalkyl;
R¹ and R² may be taken together to form a 5 to 7 membered saturated or unsaturated carbocyclic ring optionally containing 0 to 3 heteroatoms selected from the group consisting of N, O, or S, and wherein said ring is optionally substituted with R¹;
R³ is selected from the group consisting of: alkylamino, CONHR⁷, NH₂, NHCOR⁶, and CH₂NHCOR⁶;
R⁴ is selected from the group consisting of: halogen, alkylsulfinyl, alkylsulfonyl, cyano, alkoxycarbonyl, alkyl, haloalkyl, hydrido, hydroxyalkyl, haloalkoxy, heterocyclic, nitro, acylamino, aryl, heteroaryl, and alkenyl, OR¹³, SR⁸, SO₂N(R⁸)R^{8′}, NHR⁹, NHCOR⁹, NR⁹COR⁹, NHCO(OR⁹), NR⁹CO(OR⁹), NR⁸SO₂R¹⁰, NHSO₂N(R¹⁰)R^{10′}, NR⁶CON(R¹⁰)R^{10′}, COR⁹, CO₂R⁸, CON(R⁸)R^{8′}, wherein R⁸ and R^{8′} may be taken together to form a 3-7 membered carbocyclic ring having 1 to 3 substituted or unsubstituted heteroatoms selected from S, SO, SO₂, O, N, and NR⁶, and wherein
R¹⁰ and R^{10′} may be taken together to form a 3-7 membered carbocyclic ring having 1 to 3 substituted or unsubstituted heteroatoms selected from S, SO, SO₂, O, N, and NR⁶ wherein said aryl, heterocyclic, heteroaryl, or alkenyl are optionally substituted with R⁹;
R⁵ is selected from the group consisting of: hydrido, alkyl, aryl, arylalkyl, heteroaryl, heterocyclicalkyl, and heteroarylalkyl, wherein aryl, alkyl, arylalkyl, heteroaryl, heterocyclicalkyl, or heteroarylalkyl are optionally substituted with one or more radicals selected from the group consisting of OR¹⁴, N(R¹⁴)R^{14′}, and glycols;
R⁶ is independently selected from the group consisting of: hydrido, aryl, heteroaryl, lower alkyl, haloalkyl, alkenyl, alkynyl, hydroxyalkyl, aminoalkyl, alkylaminoalkyl, alkoxy, alkoxyalkyl, heterocyclicalkyl, and heterocyclic;
R⁷ is independently selected from the group consisting of: hydrido, aryl, heteroaryl, lower alkyl, haloalkyl, alkenyl, alkynyl, hydroxyalkyl, aminoalkyl, alkylaminoalkyl, alkoxy, alkoxyalkyl, heterocyclicalkyl, and heterocyclic;
R⁸ is independently selected from the group consisting of: hydrido, aryl, heteroaryl, arylalkyl, heterocyclic, haloalkyl, arylalkylamino, alkylaminoalkyl, dialkylaminoalkyl, alkyl, alkenyl, alkynyl, heteroarylalkyl, and heterocyclicalkyl;
R^{8′} is independently selected from the group consisting of: hydrido, aryl, heteroaryl, arylalkyl, heterocyclic, haloalkyl, arylalkylamino, alkylaminoalkyl, dialkylaminoalkyl, alkyl, alkenyl, alkynyl, heteroarylalkyl, and heterocyclicalkyl;
R⁹ is independently selected from the group consisting of: hydrido, lower alkyl, aryl, heteroaryl, arylalkyl, heterocyclic, cycloalkyl, heterocyclicalkyl, haloalkyl, arylalkylamino, amino, aminoalkyl, aminoacyl, nitro, azido, and heteroarylalkyl, wherein alkyl, aryl, heteroaryl, aminoalkyl, or arylalkyl are optionally substituted with one or more radical selected from the group consisting of: alkylsulfonamide, sulfamyl, alkyl, alkylthio, alkylsulfinyl, alkylsulfonyl, alkylamino, aminoalkyl, alkylaminoalkyl, alkoxy, halogen, acyloxy, oxy, formyl, haloalkyl, cyano, haloalkoxy, acyl, carboxyl, hydroxy, hydroxyalkyloxy, phenoxy, nitro, azido, benzyloxy, dialkylaminoacyl, thioalkyl, aminoacyloxy, thiocyanate, isothiocyanate, alkyldioxy, hydroxyalkyl, alkylamino, alkyloxycarbonyl, alkoxyalkyl, alkenylamino, alkynylamino, alkenyl, alkynyl, dialkylaminoalkyloxy, and heterocyclic optionally substituted with alkyl, alkylamino, aminoalkyl, and alkylaminoalkyl;
R¹⁰ is independently selected from the group consisting of: hydrido, lower alkyl, heteroaryl, heterocyclic, haloalkyl, arylalkylamino, heteroarylalkyl, aryl, and arylalkyl, wherein aryl, heteroaryl, heterocyclic, or arylalkyl are optionally substituted with one or more radical selected from alkyl, alkoxy, halogen, haloalkyl, cyano, haloalkoxy, acyl, carboxyl, hydroxy, hydroxyalkyloxy, phenoxy, benzyloxy, dialkylaminoalkyloxy, and heterocyclic,
R^{10′} is independently selected from the group consisting of: hydrido, lower alkyl, heteroaryl, heterocyclic, haloalkyl, arylalkylamino, heteroarylalkyl, aryl, and arylalkyl, wherein aryl, heteroaryl, heterocyclic, or arylalkyl are optionally substituted with one or more radical selected from alkyl, alkoxy, halogen, haloalkyl, cyano, haloalkoxy, acyl, carboxyl, hydroxy, hydroxyalkyloxy, phenoxy, benzyloxy, dialkylaminoalkyloxy, and heterocyclic,
R¹¹ is selected from the group consisting of: hydrido, halogen, haloalkyl, CN, CO₂R⁵, lower alkyl, lower alkenyl, lower alkynyl, alkoxy, and CONH₂;
R¹² is hydrido;
R¹³ is selected from the group consisting of: hydrido, alkyl, aryl, arylalkyl, heteroaryl, heterocyclicalkyl, and heteroarylalkyl, wherein aryl, alkyl, arylalkyl, heteroaryl, heterocyclicalkyl, or heteroarylalkyl are optionally substituted with one or more radicals selected from the group consisting of OR¹⁴, N(R¹⁴)R^{14′}, and glycols;
R¹⁴ is independently selected from the group consisting of hydrido, and lower alkyl; and
R^{14′} is independently selected from the group consisting of hydrido, and lower alkyl;
or isomers, tautomers, carriers, esters, prodrugs, pharmaceutically acceptable salts thereof.

6. The compound of claim 5
R¹ is selected from the group consisting of: SO₂R⁶, NR⁶R⁷, NR⁶SO₂R⁷, and SO₂N(R⁶)R⁷ wherein R⁶ and R⁷ may be taken together to form a 3-7 membered carbocyclic ring having 1 to 3 substituted or unsubstituted heteroatoms selected from the group consisting of: S, SO, SO₂, O, and NR⁶;
R² is hydrido;
R³ is selected from the group consisting of: CONHR⁷, NHCOR⁶, and CH₂NHCOR⁶;
R⁴ is selected from the group consisting of: halogen, alkylsulfinyl, alkylsulfonyl, cyano, alkoxycarbonyl, alkyl, haloalkyl, hydrido, hydroxyalkyl, haloalkoxy, heterocyclic, nitro, acylamino, aryl, heteroaryl, and alkenyl, OR¹³, SR⁸, SO₂N(R⁸)R^{8′}, NHR⁹, NHCOR⁹, NR⁹COR⁹, NHCO(OR⁹), NR⁹CO(OR⁹), NR⁸SO₂R¹⁰, NHSO₂N(R¹⁰)R^{10′}, NR⁶CON(R¹⁰)R^{10′}, COR⁹, CO₂R⁸, CON(R⁸)R^{8′}, wherein R⁸ and R^{8′} may be taken together to form a 3-7 membered carbocyclic ring having 1 to 3 substituted or unsubstituted heteroatoms selected from S, SO, SO₂, O, N, and NR⁶, and wherein R¹⁰ and R^{10′} may be taken together to form a 3-7 membered carbocyclic ring having 1 to 3 substituted or unsubstituted heteroatoms selected from S, SO, SO₂, O, N, and NR⁶ wherein said aryl, heterocyclic, heteroaryl, or alkenyl are optionally substituted with R⁹;
R⁵ is selected from the group consisting of: hydrido, alkyl, aryl, arylalkyl, heteroaryl, heterocyclicalkyl, and heteroarylalkyl, wherein aryl, alkyl, arylalkyl, heteroaryl, heterocyclicalkyl, or heteroarylalkyl are optionally substituted with one or more radicals selected from the group consisting of OR¹⁴, N(R¹⁴)R^{14′}, and glycols;
R⁶ is independently selected from the group consisting of: hydrido, aryl, heteroaryl, lower alkyl, haloalkyl, alkenyl, alkynyl, hydroxyalkyl, aminoalkyl, alkylaminoalkyl, alkoxy, alkoxyalkyl, heterocyclicalkyl, and heterocyclic;
R⁷ is independently selected from the group consisting of: hydrido, aryl, heteroaryl, lower alkyl, haloalkyl, alkenyl, alkynyl, hydroxyalkyl, aminoalkyl, alkylaminoalkyl, alkoxy, alkoxyalkyl, heterocyclicalkyl, and heterocyclic;
R⁸ is independently selected from the group consisting of: hydrido, aryl, heteroaryl, arylalkyl, heterocyclic, haloalkyl, arylalkylamino, alkylaminoalkyl, dialkylaminoalkyl, alkyl, alkenyl, alkynyl, heteroarylalkyl, and heterocyclicalkyl;
R^{8′} is independently selected from the group consisting of: hydrido, aryl, heteroaryl, arylalkyl, heterocyclic, haloalkyl, arylalkylamino, alkylaminoalkyl, dialkylaminoalkyl, alkyl, alkenyl, alkynyl, heteroarylalkyl, and heterocyclicalkyl;
R⁹ is independently selected from the group consisting of: hydrido, lower alkyl, aryl, heteroaryl, arylalkyl, heterocyclic, cycloalkyl, heterocyclicalkyl, haloalkyl, arylalkylamino, amino, aminoalkyl, aminoacyl, nitro, azido, and heteroarylalkyl, wherein alkyl, aryl, heteroaryl, aminoalkyl, or arylalkyl are optionally substituted with one or more radical selected from the group consisting of: alkylsulfonamide, sulfamyl, alkyl, alkylthio, alkylsulfinyl, alkylsulfonyl, alkylamino, aminoalkyl, alkylaminoalkyl, alkoxy, halogen, acyloxy, oxy, formyl, haloalkyl, cyano, haloalkoxy, acyl, carboxyl, hydroxy, hydroxyalkyloxy, phenoxy, nitro, azido, benzyloxy, dialkylaminoacyl, thioalkyl, aminoacyloxy, thiocyanate, isothiocyanate, alkyldioxy, hydroxyalkyl, alkylamino, alkyloxycarbonyl, alkoxyalkyl, alkenylamino, alkynylamino, alkenyl, alkynyl, dialkylaminoalkyloxy, and heterocyclic optionally substituted with alkyl, alkylamino, aminoalkyl, and alkylaminoalkyl;
R¹⁰ is independently selected from the group consisting of: hydrido, lower alkyl, heteroaryl, heterocyclic, haloalkyl, arylalkylamino, heteroarylalkyl, aryl, and arylalkyl, wherein aryl, heteroaryl, heterocyclic, or arylalkyl are optionally substituted with one or more radical selected from alkyl, alkoxy, halogen, haloalkyl, cyano, haloalkoxy, acyl, carboxyl, hydroxy, hydroxyalkyloxy, phenoxy, benzyloxy, dialkylaminoalkyloxy, and heterocyclic,
R^{10′} is independently selected from the group consisting of: hydrido, lower alkyl, heteroaryl, heterocyclic, haloalkyl, arylalkylamino, heteroarylalkyl, aryl, and arylalkyl, wherein aryl, heteroaryl, heterocyclic, or arylalkyl are optionally substituted with one or more radical selected from alkyl, alkoxy, halogen, haloalkyl, cyano, haloalkoxy, acyl, carboxyl, hydroxy, hydroxyalkyloxy, phenoxy, benzyloxy, dialkylaminoalkyloxy, and heterocyclic,
R¹¹ is selected from the group consisting of: hydrido, halogen, haloalkyl, CN, CO₂R⁵, lower alkyl, lower alkenyl, lower alkynyl, alkoxy, and CONH₂;
R¹² is hydrido;
R¹³ is selected from the group consisting of: hydrido, alkyl, aryl, arylalkyl, heteroaryl, heterocyclicalkyl, and heteroarylalkyl, wherein aryl, alkyl, arylalkyl, heteroaryl, heterocyclicalkyl, or heteroarylalkyl are optionally substituted with one or more radicals selected from the group consisting of OR¹⁴, N(R¹⁴)R^{14′}, and glycols;
R¹⁴ is independently selected from the group consisting of hydrido, and lower alkyl; and
R^{14′} is independently selected from the group consisting of hydrido, and lower alkyl;
or isomers, tautomers, carriers, esters, prodrugs, pharmaceutically acceptable salts thereof.

7. The compound of claim 6
wherein
R¹ is selected from the group consisting of: SO₂NH₂, SO₂NR⁶R⁷, SO₂R⁶;
R³ is CONH₂;
R⁴ is selected from the group consisting of: hydrido, halogen, lower alkyl, aryl, substituted aryl, heteroaryl, substituted heteroaryl, arylalkyl, heteroaryl alkyl, and alkoxy;
R⁶ is independently selected from the group consisting of: hydrido, aryl, heteroaryl, lower alkyl, haloalkyl, alkenyl, alkynyl, hydroxyalkyl, aminoalkyl, alkylaminoalkyl, alkoxy, alkoxyalkyl, heterocyclicalkyl, and heterocyclic;
R⁷ is independently selected from the group consisting of: hydrido, aryl, heteroaryl, lower alkyl, haloalkyl, alkenyl, alkynyl, hydroxyalkyl, aminoalkyl, alkylaminoalkyl, alkoxy, alkoxyalkyl, heterocyclicalkyl, and heterocyclic;
or isomers, tautomers, carriers, prodrugs, pharmaceutically acceptable salts thereof.

8. The compound of claim 7 of the formula 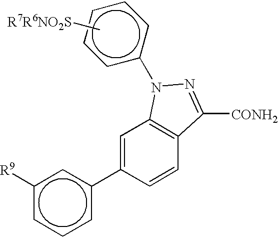
wherein
R⁶ is independently selected from the group consisting of: hydrido, aryl, heteroaryl, lower alkyl, haloalkyl, alkenyl, alkynyl, hydroxyalkyl, aminoalkyl, alkylaminoalkyl, alkoxy, alkoxyalkyl, heterocyclicalkyl, and heterocyclic;
R⁷ is independently selected from the group consisting of: hydrido, aryl, heteroaryl, lower alkyl, haloalkyl, alkenyl, alkynyl, hydroxyalkyl, aminoalkyl, alkylaminoalkyl, alkoxy, alkoxyalkyl, heterocyclicalkyl, and heterocyclic;
R⁹ is independently selected from the group consisting of: hydrido, lower alkyl, aryl, heteroaryl, arylalkyl, heterocyclic, cycloalkyl, heterocyclicalkyl, haloalkyl, arylalkylamino, amino, aminoalkyl, aminoacyl, nitro, azido, and heteroarylalkyl, wherein alkyl, aryl, heteroaryl, aminoalkyl, or arylalkyl are optionally substituted with one or more radical selected from the group consisting of: alkylsulfonamide, sulfamyl, alkyl, alkylthio, alkylsulfinyl, alkylsulfonyl, alkylamino, aminoalkyl, alkylaminoalkyl, alkoxy, halogen, acyloxy, oxy, formyl, haloalkyl, cyano, haloalkoxy, acyl, carboxyl, hydroxy, hydroxyalkyloxy, phenoxy, nitro, azido, benzyloxy, dialkylaminoacyl, thioalkyl, aminoacyloxy, thiocyanate, isothiocyanate, alkyldioxy, hydroxyalkyl, alkylamino, alkyloxycarbonyl, alkoxyalkyl, alkenylamino, alkynylamino, alkenyl, alkynyl, dialkylaminoalkyloxy, and heterocyclic optionally substituted with alkyl, alkylamino, aminoalkyl, and alkylaminoalkyl;
or isomers, tautomers, carriers, esters, prodrugs, pharmaceutically acceptable salts thereof.

9. The compound of claim 3 selected from the group consisting of:
1-[4-(aminosulfonyl)phenyl]-6-(4-methoxyphenyl)-1H-indazole-3-carboxamide,
1-[4-(aminosulfonyl)phenyl]-6-(4-fluorophenyl)-1H-indazole-3-carboxamide,
1-[4-(aminosulfonyl)phenyl]-6-(3-methylphenyl)-1H-indazole-3-carboxamide,
1-[4-(aminosulfonyl)phenyl]-6-(4-tert-butylphenyl)1-H-indazole-3-carboxamide,
1-[4-(aminosulfonyl)phenyl]-6-(4-fluoro-3-methylphenyl)-1H-indazole-3-carboxamide,
1-[4-(aminosulfonyl)phenyl]-6-[3-(dimethylamino)phenyl]-1H-indazole-3-carboxamide,
1-[4-(aminosulfonyl)phenyl]-6-[3-(methylamino)phenyl]-1H-indazole-3-carboxamide,
1-[4-(aminosulfonyl)phenyl]-1H-pyrazolo[4,3-c]pyridine-3-carboxamide,
1-[4-(aminosulfonyl)phenyl]-6-methyl-1H-indazole-3-carboxamide,
1-[4-(aminosulfonyl)phenyl]-6-phenyl-1H-indazole-3-carboxamide,
1-[4-(aminosulfonyl)phenyl]-6-(3-methoxyphenyl)-1H-indazole-3-carboxamide,
1-[4-(aminosulfonyl)phenyl]-6-benzyl-1H-indazole-3-carboxamide,
1-[4-(aminosulfonyl)phenyl]-6-ethoxy-1H-indazole-3-carboxamide,
1-[4-(aminosulfonyl)phenyl]-6-ethyl-1H-indazole-3-carboxamide,
1-[4-(aminosulfonyl)phenyl]-6-pyridin-3-yl-1H-indazole-3-carboxamide,
1-[4-(aminosulfonyl)phenyl]-6-(2-hydroxyphenyl)-1H-indazole-3-carboxamide,
1-[4-(aminosulfonyl)phenyl]-6-(3-hydroxyphenyl)-1H-indazole-3-carboxamide,
6-(2-hydroxyphenyl)-1-[4-(methylsulfonyl)phenyl]-1H-indazole-3-carboxamide, and
1-[3-(aminosulfonyl)phenyl]-6-phenyl-1H-indazole-3-carboxamide.

10. The compound of claim 6 of the formula 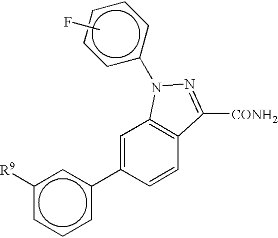
wherein
R⁹ is independently selected from the group consisting of: hydrido, lower alkyl, aryl, heteroaryl, arylalkyl, heterocyclic, cycloalkyl, heterocyclicalkyl, haloalkyl, arylalkylamino, amino, aminoalkyl, aminoacyl, nitro, azido, and heteroarylalkyl, wherein alkyl, aryl, heteroaryl, aminoalkyl, or arylalkyl are optionally substituted with one or more radical selected from the group consisting of: alkylsulfonamide, sulfamyl, alkyl, alkylthio, alkylsulfinyl, alkylsulfonyl, alkylamino, aminoalkyl, alkylaminoalkyl, alkoxy, halogen, acyloxy, oxy, formyl, haloalkyl, cyano, haloalkoxy, acyl, carboxyl, hydroxy, hydroxyalkyloxy, phenoxy, nitro, azido, benzyloxy, dialkylaminoacyl, thioalkyl, aminoacyloxy, thiocyanate, isothiocyanate, alkyldioxy, hydroxyalkyl, alkylamino, alkyloxycarbonyl, alkoxyalkyl, alkenylamino, alkynylamino, alkenyl, alkynyl, dialkylaminoalkyloxy, and heterocyclic optionally substituted with alkyl, alkylamino, aminoalkyl, and alkylaminoalkyl;
or isomers, tautomers, carriers, esters, prodrugs, pharmaceutically acceptable salts thereof.

11. The compound of claim 6 of the formula 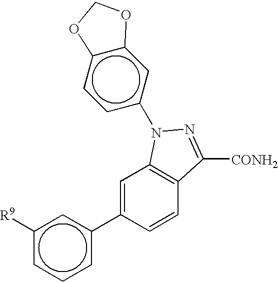
wherein
R⁹ is independently selected from the group consisting of: hydrido, lower alkyl, aryl, heteroaryl, arylalkyl, heterocyclic, cycloalkyl, heterocyclicalkyl, haloalkyl, arylalkylamino, amino, aminoalkyl, aminoacyl, nitro, azido, and heteroarylalkyl, wherein alkyl, aryl, heteroaryl, aminoalkyl, or arylalkyl are optionally substituted with one or more radical selected from the group consisting of: alkylsulfonamide, sulfamyl, alkyl, alkylthio, alkylsulfinyl, alkylsulfonyl, alkylamino, aminoalkyl, alkylaminoalkyl, alkoxy, halogen, acyloxy, oxy, formyl, haloalkyl, cyano, haloalkoxy, acyl, carboxyl, hydroxy, hydroxyalkyloxy, phenoxy, nitro, azido, benzyloxy, dialkylaminoacyl, thioalkyl, aminoacyloxy, thiocyanate, isothiocyanate, alkyldioxy, hydroxyalkyl, alkylamino, alkyloxycarbonyl, alkoxyalkyl, alkenylamino, alkynylamino, alkenyl, alkynyl, dialkylaminoalkyloxy, and heterocyclic optionally substituted with alkyl, alkylamino, aminoalkyl, and alkylaminoalkyl;
or isomers, tautomers, carriers, esters, prodrugs, pharmaceutically acceptable salts thereof.

12. A composition comprising the compound of claim 1 or 2, and at least one pharmaceutically acceptable carrier.

13. A method of treating cancer, inflammation or an inflammation associated disorder in a subject, said method comprising administering to the subject having or susceptible to such cancer, inflammation or inflammation associated disorder, a therapeutically-effective amount of a compound of claim 1, or 2.

14. The method of claim 11 for use in the treatment of cancer.

15. The method of claim 11 for use in the treatment of inflammation.

16. The method of claim 11 for use in the treatment of an inflammation-associated disorder.

17. The method of claim 14 wherein the inflammation-associated disorder is arthritis.

18. The method of claim 14 wherein the inflammation-associated disorder is pain

19. The method of claim 14 wherein the inflammation-associated disorder is fever.
